SEQUENCE LISTING (3 sequences)

SEQ ID NO: 1 - protein, 4 aa, Artificial Sequence
  synthetic proteasome inhibitor
IITL

SEQ ID NO: 2 - protein, 4 aa, Artificial Sequence
  synthetic proteasome inhibitor
IEAL

SEQ ID NO: 3 - protein, 4 aa, Artificial Sequence
  synthetic proteasome inhibitor
YLLL

(19) United States
(12) Patent Application Publication
(10) Pub. No.: US 2009/0105200 A1
(43) Pub. Date: Apr. 23, 2009

(54) Combination therapy

(76) Inventors: Mitchell Keegan, Marlborough, MA (US); Steven Grant, Richmond, VA (US)

Correspondence Address: CHOATE, HALL & STEWART LLP, TWO INTERNATIONAL PLACE, BOSTON, MA, 02110, US

(21) Appl. No.: 12/009,867

(22) Filed: Jan. 23, 2008

Related U.S. Application Data
(60) Provisional application No. 60/886,169, filed on Jan. 23, 2007.
(60) Provisional application No. 61/005,774, filed on Dec. 7, 2007.

(51) Int. Cl. A61K 31/56; A61K 31/395; C12N 5/02; A61P 35/04; A61K 31/4965

(52) U.S. Cl. 514/171; 514/431; 514/255.06; 435/375

(57) ABSTRACT

The invention provides a combination therapy for treating cancer and other neoplasms including romidepsin and a proteasome inhibitor. When administered together, romidepsin and a proteasome inhibitor (e.g., bortezomib) interact synergistically to selectively kill malignant cells at low (nanomolar) concentrations. The effect is particularly pronounced in malignant hematological cells (e.g., leukemia, lymphoma, multiple myeloma). The combination has also been found useful in treating bortezomib-resistant cancers and steroid-resistant cancers. The invention provides methods of killing malignant cells in vitro and in vivo. Pharmaceutical compositions, preparations, and kits including romidepsin and a proteasome inhibitor are also provided.

### RELATED APPLICATIONS

The present application claims priority under 35 U.S.C. § 119(e) to U.S. provisional applications, U.S. Ser. No. 60/886,169, filed Jan. 23, 2007, and U.S. Ser. No. 61/005,774, filed Dec. 7, 2007, each of which is incorporated herein by reference.

### BACKGROUND OF THE INVENTION

Romidepsin is a natural product which was isolated from Chromobacterium violaceum by Fujisawa Pharmaceuticals. See Published Japanese Patent Application Hei 7 (1995)-64872; U.S. Pat. No. 4,977,138, issued Dec. 11, 1990, which is incorporated herein by reference. It is a bicyclic peptide consisting of four amino acid residues (D-valine, D-cysteine, dehydrobutyrine, and L-valine) and a novel acid (3-hydroxy-7-mercapto-4-heptenoic acid). Romidepsin is a depsipeptide which contains both amide and ester bonds. In addition to fermentation from C. violaceum, romidepsin can also be prepared by synthetic or semi-synthetic means. The total synthesis of romidepsin reported by Kahn et al. involves 14 steps and yields romidepsin in 18% overall yield. J. Am. Chem. Soc. 118:7237-7238, 1996. The structure of romidepsin is shown below:

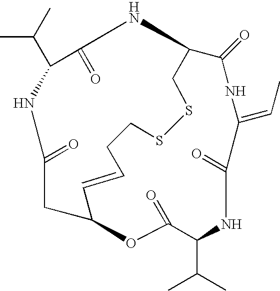

Romidepsin has been shown to have anti-microbial, immunosuppressive, and anti-tumor activities. It is thought to act by selectively inhibiting deacetylases (e.g., histone deacetylase (HDAC), tubulin deacetylase (TDAC)), promising new targets for the development of anti-cancer therapies. Nakajima et al., Experimental Cell Res. 241:126-133, 1998. One mode of action is thought to involve the inhibition of one or more classes of histone deacetylases (HDAC).

Histone deacetylase is a metallodeacetylation enzyme having zinc in its active site. Finnin et al., Nature, 401:188-193, 1999. This enzyme is thought to regulate gene expression by enhancing the acetylation of histones, thereby inducing chromatin relaxation and generally, but not universally, transcriptional activation. Although these enzymes are known as HDACs, they have also been implicated in various other cellular processes. For example, HDAC inhibitors have been found to trigger apoptosis in tumor cells through diverse mechanisms, including the up-regulation of death receptors, Bid cleavage, ROS generation, Hsp90 dysregulation, and ceramide generation, among others. Several HDAC inhibitors have entered the clinical arena and are demonstrating activity in both hematologic and non-hematologic malignancies. Romidepsin has shown impressive activity in certain hematologic malignancies, particularly T-cell lymphoma (Piekarz et al. “A review of depsipeptide and other histone deacetylase inhibitors in clinical trials” Curr. Pharm. Des. 10:2289-98, 2004; incorporated herein be reference).

In addition to romidepsin, various derivatives have been prepared and studied. The following patents and patent applications describe various derivatives of romidepsin: U.S. Pat. No. 6,548,479; WO 05/0209134; WO 05/058298; and WO 06/129105; each of which is incorporated herein by reference.

The proteasome inhibitor, bortezomib (VELCADE®) is a potent inhibitor of the catalytic unit of the 26S proteasome. It induces apoptosis in various neoplastic cell lines while exerting relatively little toxicity toward normal cells. The mechanism of bortezomib's lethality is not known with certainty but has been attributed inter alia to the inhibition of NF-κB, secondary to sparing of the NF-κB inhibitor IκBα from proteasomal degradation. Bortezomib is highly active in multiple myeloma and has been approved for use in myeloma patients refractory to standard therapy. Recent studies indicate that it also appears to be active against several forms of non-Hodgkin's lymphoma, including mantle cell lymphoma.

### SUMMARY OF THE INVENTION

The present invention provides a treatment for proliferative diseases such as cancer and other neoplasms using a combination of romidepsin and a proteasome inhibitor (e.g. bortezomib). The invention stems from the recognition that when both of these pharmaceutical agents are administered together there is an unexpected synergy between the two pharmaceutical agents. That is, lower doses of these drugs than are typically used when each agent is used individually can be administered and still be effective at treating the subject's cancer or other neoplasms. This synergistic effect is particularly pronounced in treating malignancies of hematological cells. For example, the combination is shown herein to be particularly effective in treating multiple myeloma and chronic lymphocytic leukemia (CLL). In certain embodiments, such low dose combinations are more cytotoxic to neoplastic cells than to normal cells. The inventive combination therapy has been found to be particularly useful in treating refractory and/or recurrent cancers (e.g., bortezomib-resistant cancers, steroid-resistant cancers). The invention not only provides methods of using the inventive combination of agents but also includes pharmaceutical compositions and kits including the inventive combination.

In one aspect, the invention provides a method of treating cancer in a subject (e.g., human) by administering therapeutically effective amounts of romidpesin and a proteasome inhibitor to the subject. In certain embodiments, the combination includes romidepsin and bortezomib. Both of these agents have been used in the clinic to treat human subjects with cancer. In certain embodiments, the romidpesin and bortezomib may be used in combination at dosages lower than when each is used individually. In other embodiments, the additive nature of the combination is particularly useful in treating cancer or other neoplasms. In certain embodiments, the romidpesin is administered at a dosage of 0.5 mg/m² to 15 mg/m², and bortezomib is administered at a dosage of 0.1 mg/m² to 5 mg/m². The two drugs may be administered together, or one after another. The method is particular useful in treating hematological malignancies (e.g., multiple myeloma, CLL). In certain embodiments, the cancer is resistant to bortezomib. In certain embodiments, the cancer is resistant to steroid treatment. In certain embodiments, the romidpesin and a proteasome inhibitor are administered in conjunction with another anti-neoplastic agent or a steroidal agent. In one particular embodiment, the romidpesin and a proteasome inhibitor are administered in conjunction with a steroidal agent (e.g., prednisolone, dexamethasone). In certain embodiments, the steroidal agent is administered at a dosage ranging from 0.25 mg to 100 mg, or from 5 mg to 60 mg, or from 10 mg to 50 mg. In a particular embodiment, the steroidal agent is administered at a dosage of approximately 40 mg. In another particular embodiment, the steroidal agent is administered at a dosage of approximately 20 mg. In certain embodiments, the romidepsin and the proteasome inhibitor are administered intravenously. In certain embodiments, each of the romidepsin and the proteasome inhibitor is administered bimonthly, monthly, triweekly, biweekly, weekly, twice a week, daily, or at variable intervals. In certain embodiments, the romidepsin is administered weekly, and the proteasome inhibitor is administered twice a week. In certain embodiments, the steroidal agent is administered bimonthly, monthly, triweekly, biweekly, weekly, twice a week, four times a week, daily, or at variable intervals. In certain embodiments, the steroidal agent is administered together with the romidepsin or the proteasome inhibitor. In certain embodiments, the steroidal agent is administered prior to or following the administration of romidepsin or the proteasome inhibitor. For example, the steroidal agent may be administered 5 to 7 days prior to the administration of romidepsin or the proteasome inhibitor.

In another aspect, the present invention provides a method of treating multiple myeloma in a subject (e.g., human) by administering a therapeutically effective amount of romidepsin and bortezomib to a subject with multiple myeloma. In certain embodiments, the therapeutically effective amount of romidepsin ranges from 4 mg/m² to 15 mg/m² or from 8 mg/m² to 10 mg/m². In certain embodiments, the therapeutically effective amount of bortezomib (VELCADE®) ranges from 0.5 mg/m² to 3 mg/m². In certain embodiments, therapeutically effective amount of bortezomib (VELCADE®) is approximately 1.3 mg/m². In certain embodiments, the therapeutically effective amount of romidepsin ranges from 8 mg/m to 10 mg/m², and the therapeutically effective amount of bortezomib (VELCADE®) is approximately 1.3 mg/m². In certain embodiments, the romidepsin is administered weekly, and the bortezomib (VELCADE®) is administered twice a week. In some embodiments, the romidpesin and the bortezomib (VELCADE®) are administered in conjunction with a steroidal agent (e.g., prednisolone, dexamethasone). In certain embodiments, the steroidal agent is administered at a dosage ranging from 0.25 mg to 100 mg, or from 5 mg to 60 mg, or from 10 mg to 50 mg. In a particular embodiment, the steroidal agent is administered at a dosage of approximately 40 mg. In another particular embodiment, the steroidal agent is administered at a dosage of approximately 20 mg. In certain embodiments, the steroidal agent is administered bimonthly, monthly, triweekly, biweekly, weekly, twice a week, four times a week, daily, or at variable intervals. In certain embodiments, the steroidal agent is administered together with the romidepsin or the bortezomib (VELCADE®). In certain embodiments, the steroidal agent is administered prior to or following the administration of romidepsin or bortezomib (VELCADE®). For example, the steroidal agent may be administered 5 to 7 days prior to the administration of romidepsin or bortezomib (VELCADE®).

In another aspect, the invention provides methods of treating cells in vitro by contacting cells with a combination of romidepsin and a proteasome inhibitor such as bortezomib. The cells may be treated with a sufficient concentration of the combination to kill the treated cells. In certain embodiments, a sufficient concentration of the combination is used to induce apoptosis as evidenced by changes in levels of cellular markers of apoptosis. In certain embodiments, the cells are neoplastic cells. The cells may be from human cancers or derived from cancer cell lines (e.g., multiple myeloma, CLL). In certain embodiments, the cells are hematological cells, in particular white blood cells (e.g., T-cells, B-cells, plasma cells, etc.). In certain embodiments, the cells are lymphocytes such as B-cells or T-cells. In certain embodiments, the cells are plasma cells. The cells may be at any stage of differentiation or development. In certain embodiments, the cells are resistant to bortezomib. In certain embodiments, the cells are resistant to steroidal agents (e.g., dexamethasone, prednisolone, etc.). The methods are particularly useful for assessing the cytotoxicity of a given combination under certain conditions (e.g., concentration of each agent, combination with other pharmaceutical agents). The inventive methods may be used to ascertain the susceptibility of a subject's cancer or neoplasm to the combination therapy. The inventive combination may also be used to activate the JNK pathway in a cell, down-regulate NF-κB-dependent anti-apoptotic proteins or other anti-apoptotic proteins in a cell, and/or induce cleavage of caspase-12 in a cell. Such modulation of cellular pathways by the inventive combinations may be for clinical or research purposes.

In yet another aspect, pharmaceutical compositions or preparations comprising romidepsin and a proteasome inhibitor are provided. In certain particular embodiments, the composition or preparation comprises romidepsin and bortezomib. The pharmaceutical composition includes a therapeutically effective amount of each pharmaceutical agent for the treatment of cancer (e.g., multiple myeloma, bortezomib-resistant multiple myeloma, CLL). Since there exists a synergy when the pharmaceutical agents are administered in combination, the amount of each agent may be lower than when the agents are delivered individually. The pharmaceutical composition may include other cytotoxic agents or other anti-neoplastic agents. The pharmaceutical composition may also include other agents to alleviate pain, nausea, hair loss, weight loss, weight gain, neuropathy, cardiac arrhythmias, electrolyte deficiencies or imbalances, anemia, thrombocytopenia, immunosuppression, skin conditions, or other conditions associated with cancer or the treatment of cancer. The invention also provides kits including the inventive pharmaceutical compositions in a convenient dosage form. The agents may be packaged together or separately in the kit. The kit may include multiple doses of each agent. In certain embodiments, the kits include a sufficient amount of each agent for a full course of chemotherapy in the treatment of a subject's cancer. The kit may also include excipients or devices for use in administering the inventive combination. The kit may also include instructions for administering the inventive combination.

### DEFINITIONS

Definitions of other terms used throughout the specification include:

As used herein and in the appended claims, the singular forms “a”, “an”, and “the” include the plural reference unless the context clearly indicates otherwise. Thus, for example, a reference to “a cell” includes a plurality of such cells.

“Animal”: As used herein, the term “animal” refers to any member of the animal kingdom. In some embodiments, “animal” refers to a human, at any stage of development. In some embodiments, “animal” refers to a non-human animal, at any stage of development. In some embodiments, animals include, but are not limited to, mammals, birds, reptiles, amphibians, fish, and/or worms. In certain embodiments, the non-human animal is a mammal (e.g., a rodent, a mouse, a rat, a rabbit, a monkey, a dog, a cat, a sheep, cattle, a primate, and/or a pig). In some embodiments, an animal may be a transgenic animal, genetically-engineered animal, and/or clone.

“Depsipeptide”: The term “depsipeptide”, as used herein, refers to polypeptides that contain both ester and amide bonds. Naturally occurring depsipeptides are usually cyclic. Some depsipeptides have been shown to have potent antibiotic activity. Examples of depsipeptides include actinomycin, enniatins, valinomycin, and romidepsin.

“Effective amount”: In general, the “effective amount” of an active agent or combination of agents refers to an amount sufficient to elicit the desired biological response. As will be appreciated by those of ordinary skill in this art, the effective amount of an inventive combination may vary depending on such factors as the desired biological endpoint, the pharmacokinetics of the agents being delivered, the disease being treated, the mode of administration, and the patient. For example, the effective amount of an inventive combination (e.g., romidepsin and bortezomib) is the amount that results in reducing the tumor burden, causing a remission, or curing the patient.

“Peptide” or “protein”: According to the present invention, a “peptide” or “protein” comprises a string of at least three amino acids linked together by peptide bonds. The terms “protein” and “peptide” may be used interchangeably. Peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In certain embodiments, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide. In certain embodiments, peptide refers to depsipeptide.

“Romidepsin”: The term “romidepsin”, refers to a natural product of the chemical structure:

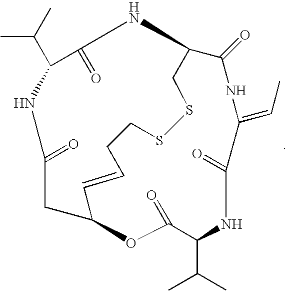

Romidepsin is a deacetylase inhibitor and is also known in the art by the names FK228, FR901228, NSC630176, or depsipeptide. The identification and preparation of romidepsin is described in U.S. Pat. No. 4,977,138, issued Dec. 11, 1990, which is incorporated herein by reference. The molecular formula is C₂₄H₃₆N₄O₆S₂; and the molecular weight is 540.71 g/mol. Romidepsin has the chemical name, (1S,4S,10S,16E,21R)-7-[(2Z)-ethylidene]-4,21-diisopropyl-2-oxa-12,13-dithia-5,8,20,23-tetraazabicyclo[8.7.6]tricos-16-ene-3,6,9,19,22-pentanone. Romidepsin has been assigned the CAS number 128517-07-7. In crystalline form, romidepsin is typically a white to pale yellowish white crystal or crystalline powder. The term “romidepsin” encompasses this compound and any pharmaceutically forms thereof. In certain embodiments, the term “romidepsin” may also include salts, pro-drugs, esters, protected forms, reduced forms, oxidized forms, isomers, stereoisomers (e.g., enantiomers, diastereomers), tautomers, and derivatives thereof.

### BRIEF DESCRIPTION OF THE DRAWING

FIG. 1. VELCADE® (Bortezomib) markedly potentiates apoptosis induced by depsipeptide in human multiple myeloma cells. Human myeloma U266 and RPM18226 cells were exposed for 48 hours to 2-3 nM romidepsin FK288 in the absence or presence of 3-4 nM bortezomib, after which the percentage of Annexin V⁺ (apoptotic) cells was determined by flow cytometry. Therefore, bortezomib and romidepsin, administered at low (nanomolar) concentrations, interact in a synergistic manner to induce apoptosis in human multiple myeloma cell lines.

FIG. 2. Romidepsin (FK288) and bortezomib interact synergistically in CD138⁺ primary human bone marrow multiple myeloma cells but not in their normal CD138⁻ counterparts. Primary CD138⁺ myeloma cells were isolated from a bone marrow sample of a patient with multiple myeloma. CD138⁺ and CD138⁻ cells were then treated for 24 hours with 3 nM romidepsin with or without 3 nM bortezomib, after which apoptosis was assessed by Annexin V-FITC staining and flow cytometry. Bortezomib and romidepsin, administered at low (nanomolar) concentrations, interact in a highly synergistic manner to induce apoptosis in primary human multiple myeloma cells, but spare normal bone marrow cells, suggesting a possible basis for therapeutic selectivity.

FIG. 3. Romidepsin (FK288)/bortezomib (Btzmb) synergistically induce apoptosis in steroid-resistant human multiple myeloma cells. Dexamethasone-sensitive (MM.1S) and -resistant (MM.1R) human myeloma cells were exposed for 24 hours to 1 nM romidepsin with and without 2 nM bortezomib, after which the percentage of Annexin V⁺ (apoptotic) cells were determined by flow cytometry. Therefore, romidepsin has been found to potentiate bortezomib lethality in steroid-resistant multiple myeloma cells when the two agents are administered at low (nanomolar) concentrations.

FIG. 4. Romidepsin (FK228) enhances the lethality of bortezomib in bortezomib-resistant U266 multiple myeloma cells. Bortezomib-resistant cells (U266/PS-R) were generated by culturing U266 cells in gradually increasing concentrations of bortezomib until a level of 12 nM was reached. U266/PS-R cells were treated for 48 hours with 2 nM romidepsin in the absence or presence of 5-15 nM bortezomib, after which apoptosis was assessed by Annexin V-FITC staining and flow cytometry. Romidepsin has been found to potentiate bortezomib lethality in multiple myeloma cells resistant to bortezomib alone.

FIG. 5. The combination of romidepsin (FK228) and bortezomib induces activation of the stress-related kinase JNK and down-regulation of NF-κB-dependent anti-apoptotic proteins in human myeloma cells. Human multiple myeloma (U266) cells were exposed for 48 hours to 2 nM romidepsin with and without 3 nM bortezomib, after which immunoblot analysis was performed to monitor JNK activation (A) and expression of NF-κB-dependent anti-apoptotic proteins (B). Synergistic interactions between romidepsin and bortezomib in human multiple myeloma cells are associated with activation of the stress-related JNK pathway and down-regulation of several NF-κB-dependent anti-apoptotic proteins (e.g., A1, Bcl-xL, and XIAP).

FIG. 6. Combined treatment with bortezomib (Btzmb) and romidepsin (FK288) administered at low concentrations (3 nM), potently induces apoptosis and caspase-12 cleavage in primary CLL cells. Romidepsin and bortezomib interact synergistically in primary, patient-derived CLL cells through a process that may involve ER stress.

FIG. 7. Photomicrographs (100×) of CLL cells exposed to bortezomib (Btzmb) with and without romidepsin (FK288). While individual treatment (3 nM each; 24 hours) is relatively non-toxic, combined treatment results in a marked increase in apoptotic cells.

FIG. 8. Romidepsin (FK228)/bortezomib interactions (48 hours) in JVM-3 and MEC-2 (pro-lymphoblastic) CLL cell lines. In each case, synergistic induction of cell death is observed.

FIG. 9. Primary CLL cells were exposed to bortezomib (Btzmb) with and without romidepsin (FK288) (3 nM each) for 24 hours, after which Western blot analysis was employed to monitor the expression of various apoptotic regulatory proteins. Combined treatment of primary CLL cells with bortezomib and romidepsin results in a marked reduction in expression of anti-apoptotic proteins, including A1, Bcl-xL, XIAP, cIAP1, ICAM-1, and c-FLIP.

FIG. 10. Clinical trial design. The clinical trial was an open label, single-centre, single-arm, phase I/II dose escalation trial of bortezomib, dexamethasone, and romidepsin in patients with relapsed or refractory multiple myeloma, followed by maintenance romidepsin therapy until disease progression.

FIG. 11. An exemplary graph summarizing individual patient treatment exposure is illustrated.

FIG. 12. Distribution of the final treatment dose level is shown. Dose level 2 is the final treatment dose level for most of the patients.

FIG. 13. Exemplary kinetics of thrombocytopaenia in patients is illustrated.

FIG. 14. A graph summarising the best response results among the patients. There were 1 immunofixation negative Complete Response (CR), 6 Partial Responses (PR), and 1 Minor Response (MR).

### DETAILED DESCRIPTION OF CERTAIN EMBODIMENTS OF THE INVENTION

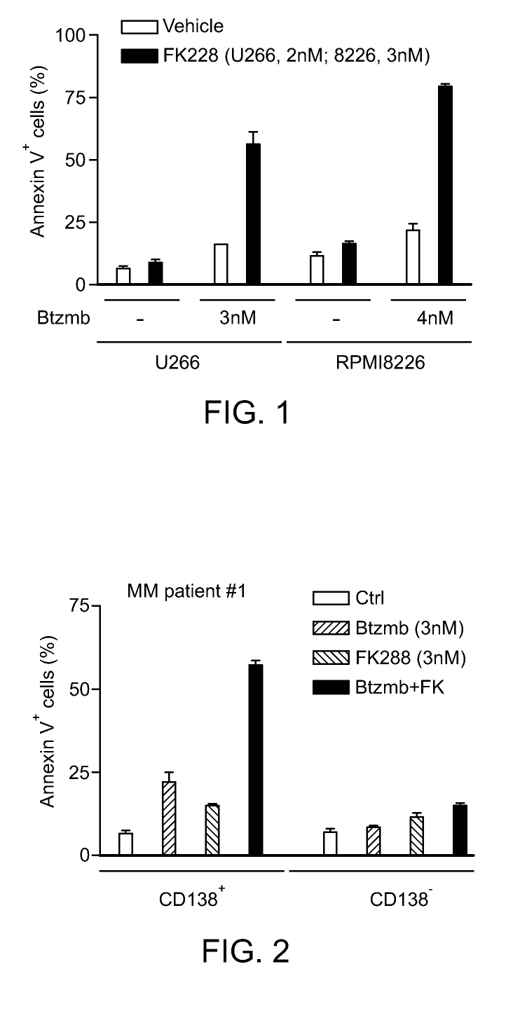

The present invention provides a novel system for treating proliferative diseases by administering a combination of romidepsin and a proteasome inhibitor. The combination of these agents may lead to an additive or synergistic effect. In certain embodiments, a synergistic interaction between romidepsin and proteasome inhibitors in the treatment of cancer or other neoplasms has been demonstrated as described herein. See FIGS. 1-9. This synergistic effect is particularly pronounced in the case of malignant hematological cells, particularly white blood cells (e.g., leukemia, lymphoma, and myeloma cells). Without wishing to be bound by any particular theory, the effect may be due to the synergistic induction of apoptosis by the combination of agents in association with the induction of mitochondrial injury and/or reactive oxygen species (ROS) generation.

One hypothesis is that interference with Hsp90 function and dynein function resulting from HDAC6-inhibitor mediated acetylation by romidepsin leads to misfolding of proteins and disordered aggresome function, which, in conjunction with proteasome inhibition, results in the potentiation of apoptosis. A second hypothesis postulates that interference with NF-κB function may contribute to the synergistic effect of the combination of romidepsin and a proteasome inhibitor. HDAC inhibitor-mediated acetylation of p65/RelA leads to NF-κB activation, which promotes HDAC inhibitor-induced ROS generation and lethality. Proteasome inhibitors such as bortezomib are thought to act analogously.

Because both romidepsin and proteasome inhibitors, such as bortezomib, may selectively target neoplastic cells, kill neoplastic cells by inducing oxidative injury, and act synergistically to trigger apoptosis in malignant cells, a combination of romidepsin and the proteasome inhibitor bortezomib was tested for its usefulness in treating cancer and found to be particularly effective as described herein. The combination of romidepsin and bortezomib has been found to be particularly useful in treating malignant hematological cells. The inventive combination is useful in treating leukemias, lymphomas, multiple myeloma, and other hematologic malignancies. The inventive combination has also been found to be useful in treating drug-resistant malignancies, such as bortezomib-resistant multiple myeloma, and steroid-resistant malignancies, such as dexamethasone-resistant multiple myeloma.

Based on these discoveries, the invention provides methods of treating cells with the inventive combinations both in vitro and in vivo. The invention also provides pharmaceutical compositions and kits comprising the inventive combinations. In certain particular embodiments, the inventive combination comprises romidepsin and bortezomib.

### Romidepsin

Romidepsin is a cyclic depsipeptide of formula:

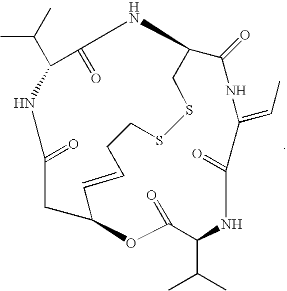

Romidepsin may be provided in any form. Pharmaceutically acceptable forms are particular preferred. Exemplary forms of romidepsin include, but are not limited to, salts, esters, pro-drugs, isomers, stereoisomers (e.g., enantiomers, diastereomers), tautomers, protected forms, reduced forms, oxidized forms, derivatives, and combinations thereof, with the desired activity (e.g., deacetylase inhibitory activity, aggresome inhibition, cytotoxicity). In certain embodiments, the romidepsin used in the combination therapy is pharmaceutical grade material and meets the standards of the U.S. Pharmacopoeia, Japanese Pharmacopoeia, or European Pharmacopoeia. In certain embodiments, the romidepsin is at least 95%, at least 98%, at least 99%, at least 99.9%, or at least 99.95% pure. In certain embodiments, the romidepsin is at least 95%, at least 98%, at least 99%, at least 99.9%, or at least 99.95% monomeric. In certain embodiments, no impurities are detectable in the romidepsin materials (e.g., oxidized material, reduced material, dimerized or oligomerized material, side products, etc.). The romidepsin typically includes less than 1.0%, less than 0.5%, less than 0.2%, or less than 0.1% of total other unknowns. The purity of romidepsin may be assessed by appearance, HPLC, specific rotation, NMR spectroscopy, IR spectroscopy, UV/Visible spectroscopy, powder x-ray diffraction (XRPD) analysis, elemental analysis, LC-mass spectroscopy, and mass spectroscopy.

The inventive combination therapy may also include a derivative of romidepsin. In certain embodiments, the derivative of romidepsin is of the formula (I):

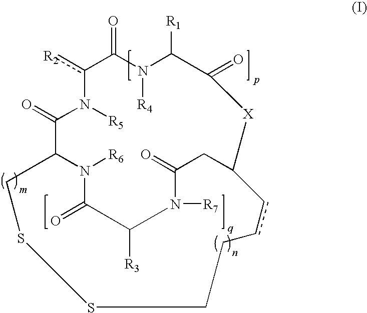

wherein

m is 1, 2, 3 or 4;

n is 0, 1, 2 or 3;

p and q are independently 1 or 2;

X is O, NH, or NR₈;

R₁, R₂, and R₃ are independently hydrogen; unsubstituted or substituted, branched or unbranched, cyclic or acyclic aliphatic; unsubstituted or substituted, branched or unbranched, cyclic or acyclic heteroaliphatic; unsubstituted or substituted aryl; or unsubstituted or substituted heteroaryl; and

R₄, R₅, R₆, R₇ and R₈ are independently hydrogen; or substituted or unsubstituted, branched or unbranched, cyclic or acyclic aliphatic; and pharmaceutically acceptable forms thereof. In certain embodiments, m is 1. In certain embodiments, n is 1. In certain embodiments, p is 1. In certain embodiments, q is 1. In certain embodiments, X is O. In certain embodiments, R₁, R₂, and R₃ are unsubstituted, or substituted, branched or unbranched, acyclic aliphatic. In certain embodiments, R₄, R₅, R₆, and R₇ are all hydrogen.

In certain embodiments, the derivative of romidepsin is of the formula (II):

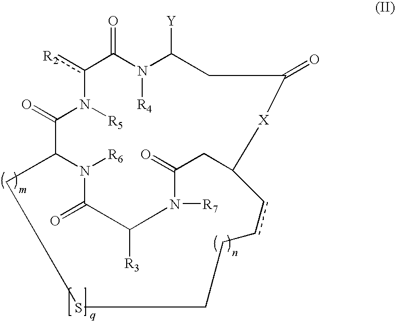

wherein:

m is 1, 2, 3 or 4;

n is 0, 1, 2 or 3;

q is 2 or 3;

X is O, NH, or NR₈;

Y is OR₈, or SR₈;

R₂ and R₃ are independently hydrogen; unsubstituted or substituted, branched or unbranched, cyclic or acyclic aliphatic; unsubstituted or substituted, branched or unbranched, cyclic or acylic heteroaliphatic; unsubstituted or substituted aryl; or unsubstituted or substituted heteroaryl;

R₄, R₅, R₆, R₇ and R₈ are independently selected from hydrogen; or substituted or unsubstituted, branched or unbranched, cyclic or acyclic aliphatic; and pharmaceutically acceptable forms thereof. In certain embodiments, m is 1. In certain embodiments, n is 1. In certain embodiments, q is 2. In certain embodiments, X is O. In other embodiments, X is NH. In certain embodiments, R₂ and R₃ are unsubstituted or substituted, branched or unbranched, acyclic aliphatic. In certain embodiments, R₄, R₅, R₆, and R₇ are all hydrogen.

In certain embodiments, the derivative of romidepsin is of the formula (III):

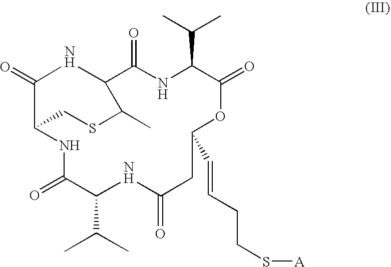

wherein

A is a moiety that is cleaved under physiological conditions to yield a thiol group and includes, for example, an aliphatic or aromatic acyl moiety (to form a thioester bond); an aliphatic or aromatic thioxy (to form a disulfide bond); or the like; and pharmaceutically acceptable forms thereof. Such aliphatic or aromatic groups can include a substituted or unsubstituted, branched or unbranched, cyclic or acyclic aliphatic group; a substituted or unsubstituted aromatic group; a substituted or unsubstituted heteroaromatic group; or a substituted or unsubstituted heterocyclic group. A can be, for example, —COR₁, —SC(═O)—O—R₁, or —SR₂. R₁ is independently hydrogen; substituted or unsubstituted amino; substituted or unsubstituted, branched or unbranched, cyclic or acyclic aliphatic; substituted or unsubstituted aromatic group; substituted or unsubstituted heteroaromatic group; or a substituted or unsubstituted heterocyclic group. In certain embodiment, R₁ is hydrogen, methyl, ethyl, n-propyl, iso-propyl, n-butyl, isobutyl, benzyl, or bromobenzyl. R₂ is a substituted or unsubstituted, branched or unbranched, cyclic or acyclic aliphatic group; a substituted or unsubstituted aromatic group; a substituted or unsubstituted heteroaromatic group; or a substituted or unsubstituted heterocyclic group. In certain embodiments, R₂ is methyl, ethyl, 2-hydroxyethyl, isobutyl, fatty acids, a substituted or unsubstituted benzyl, a substituted or unsubstituted aryl, cysteine, homocysteine, or glutathione.

In certain embodiments, the derivative of romidepsin is of formula (IV) or (IV′):

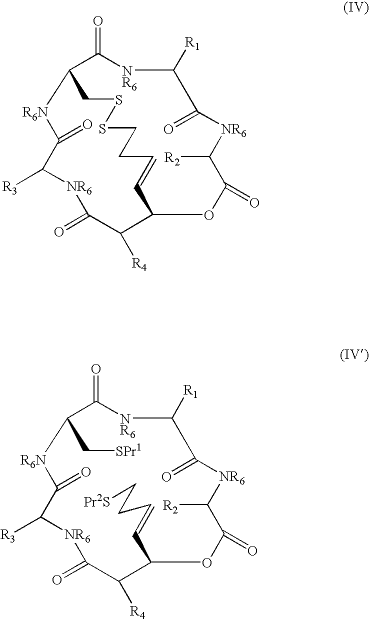

wherein

R₁, R₂, R₃, and R₄ are the same or different and represent an amino acid side chain moiety, each R₆ is the same or different and represents hydrogen or C₁-C₄ alkyl, and Pr¹ and Pr² are the same or different and represent hydrogen or thiol-protecting group. In certain embodiments, the amino acid side chain moieties are those derived from natural amino acids. In other embodiments, the amino acid side chain moieties are those derived from unnatural amino acids. In certain embodiments, each amino acid side chain is a moiety selected from —H, —C₁-C₆ alkyl, —C₂-C₆ alkenyl, -L-O—C(O)—R′, -L-C(O)—O—R″, -L-A, -L-NR″R″, -L-Het-C(O)—Het-R″, and -L-Het-R″, wherein L is a C₁-C₆ alkylene group, A is phenyl or a 5- or 6-membered heteroaryl group, each R′ is the same or different and represents C₁-C₄ alkyl, each R″ is the same or different and represent H or C₁-C₆ alkyl, each -Het- is the same or different and is a heteroatom spacer selected from —O—, —N(R′″)—, and —S—, and each R′″ is the same of different and represents H or C₁-C₄ alkyl. In certain embodiments, R₆ is —H. In certain embodiments, Pr¹ and Pr² are the same or different and are selected from hydrogen and a protecting group selected from a benzyl group which is optionally substituted by C₁-C₆ alkoxy, C₁-C₆ acyloxy, hydroxy, nitro, picolyl, picolyl-N-oxide, anthrylmethyl, diphenylmethyl, phenyl, t-butyl, adamanthyl, C₁-C₆ acyloxymethyl, C₁-C₆ alkoxymethyl, tetrahydropyranyl, benzylthiomethyl, phenylthiomethyl, thiazolidine, acetamidemethyl, benzamidomethyl, tertiary butoxycarbonyl (BOC), acetyl and its derivatives, benzoyl and its derivatives, carbamoyl, phenylcarbamoyl, and C₁-C₆ alkylcarbamoyl. In certain embodiments, Pr¹ and Pr² are hydrogen. Various romidepsin derivatives of formula (IV) and (IV′) are disclosed in published PCT application WO 2006/129105, published Dec. 7, 2006; which is incorporated herein by reference.

Processes for preparing romidepsin are known in the art. For example, exemplary processes of preparing romidepsin are described in U.S. Ser. No. 60/882,698, filed on Dec. 29, 2006; U.S. Ser. No. 60/882,704, filed on Dec. 29, 2006; and U.S. Ser. No. 60/882,712, filed on Dec. 29, 2006, the teachings of all of which are incorporated by reference herein. Since romidepsin is a natural product, it is typically prepared by isolating it from a fermentation of a microorganism that produces it. In certain embodiments, the romidepsin or a derivate thereof is purified from a fermentation, for example, of Chromobacterium violaceum. See, e.g., Ueda et al., J. Antibiot. (Tokyo) 47:301-310, 1994; Nakajima et al., Exp. Cell Res. 241:126-133, 1998; WO 02/20817; U.S. Pat. No. 4,977,138; each of which is incorporated herein by reference. In other embodiments, romidepsin or a derivative thereof is prepared by synthetic or semi-synthetic means. J. Am. Chem. Soc. 118:7237-7238, 1996; incorporated herein by reference.

The therapeutically effective amount of romidepsin included in the combination therapy will vary depending on the patient, the cancer or neoplasm being treated, stage of the cancer, pathology of the cancer or neoplasm, genotype of the cancer or neoplasm, phenotype of the cancer or neoplasm, the route of administration, etc. In certain embodiments, the romidepsin is dosed in the range of 0.5 mg/m² to 28 mg/m². In certain embodiments, the romidepsin is dosed in the range of 1 mg/m² to 25 mg/m². In certain embodiments, the romidepsin is dosed in the range of 0.5 mg/m² to 15 mg/m². In certain embodiments, the romidepsin is dosed in the range of 1 mg/m² to 15 mg/m². In certain embodiments, the romidepsin is dosed in the range of 1 mg/m² to 8 mg/m². In certain embodiments, the romidepsin is dosed in the range of 0.5 mg/m² to 5 mg/m². In certain embodiments, the romidepsin is dosed in the range of 2 mg/m² to 10 mg/m². In certain embodiments, the romidepsin is dosed in the range of 4 mg/m² to 15 mg/m². In certain embodiments, the romidepsin is dosed in the range of 8 mg/m² to 10 mg/m². In other embodiments, the dosage ranges from 10 mg/m² to 20 mg/m². In certain embodiments, the dosage ranges from 5 mg/m² to 10 mg/m². In other embodiments, the dosage ranges from 10 mg/m² to 15 mg/m². In still other embodiments, the dosage is approximately 8 mg/m². In still other embodiments, the dosage is approximately 9 mg/m². In still other embodiments, the dosage is approximately 10 mg/m². In still other embodiments, the dosage is approximately 11 mg/m². In still other embodiments, the dosage is approximately 12 mg/m². In still other embodiments, the dosage is approximately 13 mg/m². In still other embodiments, the dosage is approximately 14 mg/m². In still other embodiments, the dosage is approximately 15 mg/m². In certain embodiments, increasing doses of romidepsin are administered over the course of a cycle. For example, in certain embodiments, a dose of approximately 8 mg/m², followed by a dose of approximately 10 mg/m², followed by a dose of approximately 12 mg/m² may be administered over a cycle. As will be appreciated by one of skill in the art, depending on the form of romidepsin being administered the dosing may vary. The dosages given herein are dose equivalents with respect to the active ingredient, romidepsin. As will be appreciated by one of skill in the art, more of a salt, hydrate, co-crystal, pro-drug, ester, solute, etc. may need to be administered to deliver the equivalent number of molecules of romidepsin. In certain embodiments, romidepsin is administered intravenously. In certain embodiments, the romidepsin is administered intravenously over a 1-6 hour time frame. In certain particular embodiments, the romidepsin is administered intravenously over 3-4 hours. In certain particular embodiments, the romidepsin is administered intravenously over 5-6 hours. In certain embodiments, the romidepsin is administered one day followed by several days in which the romidepsin is not administered. In certain embodiments, the romidepsin and the proteasome inhibitor are administered together. In other embodiments, the romidpesin and the proteasome inhibitor are administered separately. For example, the administration of romidepsin and a proteasome inhibitor may be separated by one or more days. In certain embodiments, romidepsin is administered twice a week. In certain embodiments, romidepsin is administered once a week. In other embodiments, romidepsin is administered every other week. In certain embodiments, romidepsin is administered on days 1, 8, and 15 of a 28 day cycle. In certain particular embodiments, an 8 mg/m² dose of romidepsin is administered on day 1, a 10 mg/m² dose of romidepsin is administered on day 8, and a 12 mg/m² dose of romidepsin is administered on day 15. In certain embodiments, romidepsin is administered on days 1 and 15 of a 28 day cycle. The 28 day cycle may be repeated. In certain embodiments, the 28 day cycle is repeated 3-10 times. In certain embodiments, the treatment includes 5 cycles. In certain embodiments, the treatment includes 6 cycles. In certain embodiments, the treatment includes 7 cycles. In certain embodiments, the treatment includes 8 cycles. In certain embodiments, greater than 10 cycles are administered. In certain embodiments, the cycles are continued as long as the patient is responding. The therapy may be terminated once there is disease progression, a cure or remission is achieved, or side effects become intolerable.

Alternatively, romidepsin may be administered orally. In certain embodiments, romidepsin is dosed orally in the range of 10 mg/m² to 300 mg/m². In certain embodiments, romidepsin is dosed orally in the range of 25 mg/m² to 100 mg/m². In certain embodiments, romidepsin is dosed orally in the range of 100 mg/m² to 200 mg/m². In certain embodiments, romidepsin is dosed orally in the range of 200 mg/m² to 300 mg/m². In certain embodiments, dosed orally in the range of 50 mg/m² to 150 mg/m². In other embodiments, the oral dosage ranges from 25 mg/m² to 75 mg/m². As will be appreciated by one of skill in the art, depending on the form of romidepsin being administered the dosing may vary. The dosages given herein are dose equivalents with respect to the active ingredient, romidepsin. In certain embodiments, romidepsin is administered orally on a daily basis. In other embodiments, romidepsin is administered orally every other day. In still other embodiments, romidepsin is administered orally every third, fourth, fifth, or sixth day. In certain embodiments, romidepsin is administered orally every week. In certain embodiments, romidepsin is administered orally every other week. In certain embodiments, the romidepsin and the proteasome inhibitor are administered together. In other embodiments, the romidepsin and the proteasome inhibitor are administered separately. For example, the administration of romidepsin and a proteasome inhibitor may be separated by one or more days. In certain embodiments, both romidepsin and the proteasome inhibitor are administered orally. In certain embodiments, only romidepsin is administered orally. The administration of romidepsin alone or the combination of romidepsin and the proteasome inhibitor may be terminated once there is disease progression, a cure or remission is achieved, or side effects become intolerable.

### Proteasome Inhibitor

The proteasome is a multi-subunit protease that degrades most cytosolic, endoplasmic reticulum, and nuclear proteins. Kisselev and Goldberg, “Proteasome inhibitors: from research tools to drug candidates” Chem. Biol. 8:739-58, 2001; incorporated herein by reference. Ubiquitin-tagged proteins are unfolded and fed through the core of the proteasome which possesses various proteolytic active sites.

Any proteasome inhibitor may be combined with romidepsin in the treatment of cancer or other neoplasms. The proteasome inhibitor typically interacts synergistically or additively with romidepsin to kill neoplastic or malignant cells. A synergistic effect allows for the use of lower doses of the proteasome inhibitor than would normally be used if the proteasome inhibitor were administered alone in the treatment of cancer or another neoplasm. In certain embodiments, the proteasome inhibitor may interact synergistically with romidepsin to induce apoptosis in the neoplastic or malignant cells. In certain embodiments, the combination acts synergistically to induce oxidative injury.

Examples of proteasome inhibitors include bortezomib (VELCADE®), peptide boronates, salinosporamide A (NPI-0052), lactacystin, epoxomicin (Ac(Me)-Ile-Ile-Thr-Leu-EX), MG-132 (Z-Leu-Leu-Leu-al), PR-171, PS-519, eponemycin, aclacinomycin A, CEP-1612, CVT-63417, PS-341 (pyrazylcarbonyl-Phe-Leu-boronate), PSI (Z-Ile-Glu(OtBu)-Ala-Leu-al), MG-262 (Z-Leu-Leu-Leu-bor), PS-273 (MNLB), omuralide (clasto-lactacystin-β-lactone), NLVS (Nip-Leu-Leu-Leu-vinyl sulfone), YLVS (Tyr-Leu-Leu-Leu-vs), dihydroeponemycin, DFLB (dansyl-Phe-Leu-boronate), ALLN (Ac-Leu-Leu-Nle-al), 3,4-dichloroisocoumarin, 4-(2-aminoethyl)-benzenesulfonyl fluoride, TMC-95A, gliotoxin, EGCG ((−)-epigallocatechin-3-gallate), and YU101 (Ac-hFLFL-ex). In certain embodiments, romidepsin is combined with bortezomib (VELCADE®). In certain embodiments, romidepsin is combined with salinosporamide A (NPI-0052). In certain embodiments, romidepsin is combined with lactacystin. In certain embodiments, romidepsin is combined with epoxomicin (Ac(Me)-Ile-Ile-Thr-Leu-EX). In certain embodiments, romidepsin is combined with MG-132 (Z-Leu-Leu-Leu-al). In certain embodiments, romidepsin is combined with PR-171. In certain embodiments, romidepsin is combined with PS-519. In certain embodiments, romidepsin is combined with eponemycin. In certain embodiments, romidepsin is combined with aclacinomycin A. In certain embodiments, romidepsin is combined with CEP-1612. In certain embodiments, romidepsin is combined with CVT-63417. In certain embodiments, romidepsin is combined with PS-341 (pyrazylcarbonyl-Phe-Leu-boronate). In certain embodiments, romidepsin is combined with PSI (Z-Ile-Glu(OtBu)-Ala-Leu-al). In certain embodiments, romidepsin is combined with PS-341 (pyrazylcarbonyl-Phe-Leu-boronate). In certain embodiments, romidepsin is combined with MG-262 (Z-Leu-Leu-Leu-bor). In certain embodiments, romidepsin is combined with PS-273 (MNLB). In certain embodiments, romidepsin is combined with omuralide (clasto-lactacystin-β-lactone). In certain embodiments, romidepsin is combined with NLVS (Nip-Leu-Leu-Leu-vinyl sulfone). In certain embodiments, romidepsin is combined with PS-273 (MNLB). In certain embodiments, romidepsin is combined with YLVS (Tyr-Leu-Leu-Leu-vs). In certain embodiments, romidepsin is combined with dihydroeponemycin. In certain embodiments, romidepsin is combined with DFLB (dansyl-Phe-Leu-boronate). In certain embodiments, romidepsin is combined with ALLN (Ac-Leu-Leu-Nle-al). In certain embodiments, romidepsin is combined with 3,4-dichloroisocoumarin. In certain embodiments, romidepsin is combined with 4-(2-aminoethyl)-benzenesulfonyl fluoride. In certain embodiments, romidepsin is combined with TMC-95A. In certain embodiments, romidepsin is combined with gliotoxin. In certain embodiments, romidepsin is combined with EGCG ((−)-epigallocatechin-3-gallate). In certain embodiments, romidepsin is combined with YU101 (Ac-hFLFL-ex).

In certain particular embodiments, the proteasome inhibitor bortezomib is combined with romidepsin. Bortezomib has the chemical name, [(1R)-3-methyl-1-[[(2S)-1-oxo-3-phenyl-2-[(pyrazinylcarbonyl)amino]propyl]amino]butyl]boronic acid, and is of the formula:

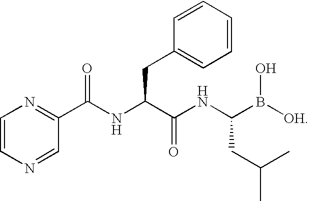

Bortezomib may be administered concurrently with romidepsin, subsequent to romidepsin, or prior to romidepsin. Bortezomib is typically administered parenterally as an intravenous bolus. In certain embodiments, the dosage of bortezomib ranges for 0.1 to 10 mg/m². In certain embodiments, the dosage of bortezomib ranges for 0.1 to 5 mg/m². In certain embodiments, the dosage ranges from 1 to 5 mg/m². In certain embodiments, the dosage ranges from 0.1 to 1 mg/m². In certain embodiments, the dosage ranges from 0.5 to 1.5 mg/m². In certain embodiments, the dosage ranges from 0.5 to 1.0 mg/m². In certain embodiments, the dosage ranges from 0.5 to 3.0 mg/m². In certain embodiments, the dosage is approximately 0.7 mg/m². In certain embodiments, the dosage is approximately 0.8 mg/m². In certain embodiments, the dosage is approximately 0.9 mg/m². In certain embodiments, the dosage is approximately 1.0 mg/m². In certain other embodiments, the dosage is approximately 1.1 mg/m². In certain other embodiments, the dosage is approximately 1.2 mg/m². In certain other embodiments, the dosage is approximately 1.3 mg/m². In certain other embodiments, the dosage is approximately 1.4 mg/m². In certain other embodiments, the dosage is approximately 1.5 mg/m². As will be appreciated by one of skill in the art, depending on the form of bortezomib being administered the dosing may vary. The dosages given herein are dose equivalents with respect to the active ingredient, bortezomib. The administration of bortezomib is typically followed by rest period. The rest period may range from 2 days to 14 days. In certain embodiments, the rest period is at least 24 hours, 48 hours, or 72 hours. In certain embodiments, the rest period is 1 week. In certain embodiments, bortezomib is administered biweekly. In certain embodiments, bortezomib is administered once weekly. In certain particular embodiments, bortezomib is administered once a week for four weeks. In other embodiments, bortezomib is administered twice weekly. In certain embodiments, bortezomib is administered twice weekly for two weeks (days 1, 4, 8, and 11) followed by a 10-day rest period. In certain embodiments, bortezomib is administered on days 1, 4, 8, and 11 of a 28 day cycle. A treatment cycle may range from 3 weeks to 10 weeks. In certain embodiments, the treatment cycle is 3 weeks. In other embodiments, the treatment cycle is 4 weeks. In other embodiments, the treatment cycle is 5 weeks. In yet other embodiments, the treatment cycle is 6 weeks. In yet other embodiments, the treatment cycle is 7 weeks. In yet other embodiments, the treatment cycle is 8 weeks. In certain embodiments, the cycles are continued as long as the patient is responding. The therapy may be terminated once there is disease progression, a cure or remission is achieved, or side effects become intolerable.

### Anti-Neoplastic Agents and Steroidal Agents

Anti-neoplastic agents suitable for the present invention includes any agents that inhibit or prevent the growth of neoplasms, checking the maturation and proliferation of malignant cells. Growth inhibition can occur through the induction of stasis or cell death in the tumor cell(s). Typically, antineoplastic agents include cytotoxic agents in general. Exemplary anti-neoplastic agents include, but are not limited to, cytokines, ligands, antibodies, radionuclides, and chemotherapeutic agents. In particular, such agents include interleukin 2 (IL-2), interferon (IFN) TNF; photosensitizers, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶ Re), and rhenium-188 (¹⁸⁸ Re); chemotherapeutics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing antitumor agents (e.g., antisense oligonucleotides, plasmids encoding toxins, methotrexate, etc.); and other antibodies or antibody fragments, such as F(ab).

Exemplary steroidal agents suitable for the present invention include, but are not limited to, alclometasone diproprionate, amcinonide, beclomethasone diproprionate, betamethasone, betamethasone benzoate, betamethasone diproprionate, betamethasone sodium phosphate, betamethasone sodium phosphate and acetate, betamethasone valerate, clobetasol proprionate, clocortolone pivalate, cortisol (hydrocortisone), cortisol (hydrocortisone) acetate, cortisol (hydrocortisone) butyrate, cortisol (hydrocortisone) cypionate, cortisol (hydrocortisone) sodium phosphate, cortisol (hydrocortisone) sodium succinate, cortisol (hydrocortisone) valerate, cortisone acetate, desonide, desoximetasone, dexamethasone, dexamethasone acetate, dexamethasone sodium phosphate, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium succinate, mometasone furoate, paramethasone acetate, prednisolone, prednisolone acetate, prednisolone sodium phosphate, prednisolone tebutate, prednisone, triamcinolone, triamcinolone acetonide, triamcinolone diacetate, and triamcinolone hexacetonide or a synthetic analog thereof, or a combination thereof. In certain embodiments, the steroidal agent suitable for the invention is dexamethasone. In certain embodiments, the steroidal agent suitable for the invention is prednisolone.

In certain embodiments, the steroidal agent is administered at a dosage ranging from 0.25 mg to 100 mg. In certain embodiments, the steroidal agent is administered at a dosage ranging from 5 mg to 60 mg. In certain embodiments, the steroidal agent is administered at a dosage ranging from 10 mg to 50 mg. In a particular embodiment, the steroidal agent is administered at a dosage of approximately 40 mg. In a particular embodiment, the steroidal agent is administered at a dosage of approximately 30 mg. In another particular embodiment, the steroidal agent is administered at a dosage of approximately 20 mg. In a particular embodiment, the steroidal agent is administered at a dosage of approximately 10 mg. In a particular embodiment, the steroidal agent is administered at a dosage of approximately 5 mg. In certain embodiments, the steroidal agent is administered concurrently with the romidepsin and/or the proteasome inhibitor. In certain embodiments, the steroidal agent is administered prior to or following the administration of romidepsin or the proteasome inhibitor. For example, the steroidal agent may be administered 5 to 7 days prior to the administration of romidepsin or the proteasome inhibitor. In certain embodiments, the steroidal agent is dexamethasone, and the dosage of dexamethasone if 20 mg.

### Uses

The combination of romidepsin and a proteasome inhibitor may be used in vitro or in vivo. The inventive combination is particularly useful in the treatment of neoplasms in vivo. However, the combination may also be used in vitro for research or clinical purposes (e.g., determining the susceptibility of a patient's disease to the inventive combination, researching the mechanism of action, elucidating a cellular pathway or process). In certain embodiments, the neoplasm is a benign neoplasm. In other embodiments, the neoplasm is a malignant neoplasm. Any cancer may be treated using the inventive combination.

In certain embodiments, the malignancy is a hematological malignancy. Hematological malignancies are types of cancers that affect the blood, bone marrow, and/or lymph nodes. Examples of hematological malignancies that may be treated using the inventive combination therapy include, but are not limited to: acute lymphoblastic leukemia (ALL), acute myelogenous leukemia (AML), chronic myelogenous leukemia (CML), chronic lymphocytic leukemia (CLL), hairy cell leukemia, Hodgkin's lymphoma, non-Hodgkin's lymphoma, cutaneous T-cell lymphoma (CTCL), peripheral T-cell lymphoma (PTCL), and multiple myeloma. In certain embodiments, the inventive combination is used to treat multiple myeloma. In certain particular embodiments, the cancer is relapsed and/or refractory multiple myeloma. In other embodiments, the inventive combination is used to treat chromic lymphocytic leukemia (CLL). In certain embodiments, the inventive combination is used to treat acute lymphoblastic leukemia (ALL). In certain embodiments, the inventive combination is used to treat acute myelogenous leukemia (AML). In certain embodiments, the cancer is cutaneous T-cell lymphoma. In other embodiments, the cancer is peripheral T-cell lymphoma.

Other cancers besides hematological malignancies may also be treated using the inventive combination. In certain embodiments, the cancer is a solid tumor. Exemplary cancers that may be treated using a combination therapy include colon cancer, lung cancer, bone cancer, pancreatic cancer, stomach cancer, esophageal cancer, skin cancer, brain cancer, liver cancer, ovarian cancer, cervical cancer, uterine cancer, testicular cancer, prostate cancer, bladder cancer, kidney cancer, neuroendocrine cancer, etc. In certain embodiments, the inventive combination is used to treat pancreatic cancer. In certain embodiments, the inventive combination is used to treat prostate cancer. In certain specific embodiments, the prostate cancer is hormone refractory prostate cancer.

The combination therapy may also be used to treated a refractory or relapsed malignancy. In certain embodiments, the cancer is a refractory and/or relapsed hematological malignancy. For example, the cancer may be resistant to a particular chemotherapeutic agent. In certain embodiments, the cancer is a bortezomib-resistant malignancy. In certain particular embodiments, the cancer is a bortezomib-resistant hematological malignancy. In certain particular embodiments, the cancer is bortezomib-resistant multiple myeloma. The combination of romidepsin and bortezomib has been found to be particularly useful in treating bortezolmib-resistant hematological malignancies such as bortezomib-resistant multiple myeloma. In other embodiments, the cancer is resistant to steroid therapy. In certain embodiments, the cancer is a hematological malignancy that is resistant steroid treatment. In certain embodiments, the cancer is steroid-resistant multiple myeloma. In certain particular embodiments, the cancer is dexamethasone-resistant multiple myeloma. In certain particular embodiments, the cancer is prednisolone-resistant multiple myeloma.

The inventive combinations of romidepsin plus a proteasome inhibitor may also be used to treat and/or kill cells in vitro. In certain embodiments, a cytotoxic concentration of the combination of agents is contacted with the cells in order to kill them. In other embodiments, a sublethal concentration of the combination of agents is used to treat the cells. In certain embodiments, the combination of agents acts additively to kill the cells. In certain embodiments, the combination of agents acts synergistically to kill the cells. Therefore, a lower concentration of one or both agents is needed to kills the cells than would be needed if either agent were used alone. In certain embodiments, the concentration of each agent ranges from 0.01 nM to 100 nM. In certain embodiments, the concentration of each agent ranges from 0.1 nM to 50 nM. In certain embodiments, the concentration of each agent ranges from 1 nM to 101 nM. In certain embodiments, the concentration of romidepsin ranges from 1 nM to 10 nM, more particularly 1 nM to 5 nM. In certain embodiments, the concentration of the proteasome inhibitor bortezomib ranges from 1 nM to 10 nM, more particularly 1 nM to 5 nM

Any type of cell may be tested or killed with the combination therapy (i.e., romidepsin and a proteasome inhibitor (e.g., bortezomib)). The cells may be derived from any animal, plant, bacterial, or fungal source. The cells may be at any stage of differentiation or development. In certain embodiments, the cells are animal cells. In certain embodiments, the cells are vertebrate cells. In certain embodiments, the cells are mammalian cells. In certain embodiments, the cells are human cells. The cells may be derived from a male or female human in any stage of development. In certain embodiments, the cells are primate cells. In other embodiments, the cells are derived from a rodent (e.g., mouse, rat, guinea pig, hamster, gerbil). In certain embodiments, the cells are derived from a domesticated animal such as a dog, cat, cow, goat, pig, etc. The cells may also be derived from a genetically engineered animal or plant, such as a transgenic mouse.

The cells used may be wild type or mutant cells. The cells may be genetically engineered. In certain embodiments, the cells are normal cells. In certain embodiments, the cells are hematological cells. In certain embodiments, the cells are white blood cells. In certain particular embodiments, the cells are precursors of white blood cells (e.g., stem cells, progenitor cells, blast cells). In certain embodiments, the cells are neoplastic cells. In certain embodiments, the cells are cancer cells. In certain embodiments, the cells are derived from a hematological malignancy. In other embodiments, the cells are derived from a solid tumor. For example, the cells may be derived from a patient's tumor (e.g., from a biopsy or surgical excision). In certain embodiments, the cells are derived from a blood sample from the subject or from a bone marrow biopsy. In certain embodiments, the cells are derived from a lymph node biopsy. Such testing for cytotoxicity may be useful in determining whether a patient will respond to a particular combination therapy. Such testing may also be useful in determining the dosage needed to treat the malignancy. This testing of the susceptibility of a patient's cancer to the combination therapy would prevent the unnecessary administration of drugs with no effect to the patient. The testing may also allow the use of lower doses of one or both of the drugs if the patient's cancer is particularly susceptible to the combination.

In other embodiments, the cells are derived from cancer cells lines. In certain embodiments, the cells are from hematological malignancies such as those discussed herein. Human leukemia cell lines include U937, HL-60, THP-1, Raji, CCRF-CEM, and Jurkat. Exemplary CLL cell lines include JVM-3 and MEC-2. Exemplary myeloma cells lines include MM1.S, MM1.R (dexamethasone-resistant), RPM18226, NCI-H929, and U266. Exemplary lymphoma cell lines includes Karpas, SUDH-6, SUDH-16, L428, KMH2, and Granta mantle lymphoma cell line. In certain embodiments, the cells are AML cells or multiple myeloma (CD138⁺) cells. In certain embodiments, the cells are hematopoietic stem or progenitor cells. For example, in certain embodiments, the cells are hematopoietic progenitor cells such as CD34⁺ bone marrow cells. In certain embodiments, the cell lines are resistant to a particular chemotherapeutic agent. In certain particular embodiments, the cell line is resistant to bortezomib. In other embodiments, the cell line is steroid-resistant (e.g., dexamethasone-resistant, prednisolone-resistant). In certain particular embodiments, the cells are steroid-resistant human multiple myeloma cells.

Various markers may be assayed for in the cells treated with the inventive combination therapy. For example, the marker Annexin V may be used to identify cells undergoing apoptosis. NF-κB-dependent anti-apoptotic proteins have been shown to be down-regulated by the combination therapy. Anti-apoptotic proteins that may be assayed for include A1, Bcl-xL, XIAP, cIAP1, ICAM-1, and c-FLIP. In certain embodiments, cells are treated with an amount of romidepsin and a proteasome inhibitor such as bortezomib effective to down-regulate NF-κB-dependent anti-apoptotic proteins (e.g., A1, Bcl-xL, XIAP, cIAP1, ICAM-1, and c-FLIP) in the cells. Protein in the JNK pathway such as p-JNK may also be assayed for since the combination of romidepsin and bortezomib has been shown to activate the stress-related JNK pathway. In certain embodiments, cells are treated with an amount of romidepsin and a proteasome inhibitor such as bortezomib effective to activate the JNK pathway in the cells. In certain embodiments, cells are treated with an amount of romidepsin and a proteasome inhibitor such as bortezomib effective to induce cleavage of caspase-12 in the cells.

### Pharmaceutical Compositions

This invention also provides pharmaceutical compositions, preparations, or kits comprising romidepsin and/or a proteasome inhibitor as described herein, which combination shows cytostatic or cytotoxic activity against neoplastic cells such as hematological malignancies. The compositions, preparations, or kits typically include amounts appropriate for the administration of romidepsin and/or the proteasome inhibitor. In certain embodiments, the romidepsin and the proteasome inhibitor are not mixed together in the same composition. For example, the two agents are not part of the same solution or powder. Typically, the two agents are kept separate in two different compositions and are delivered separately. A kit may contain a pharmaceutical composition of romidepsin and a separate pharmaceutical composition of a proteasome inhibitor. In certain particular embodiments, the pharmaceutical compositions, preparations, or kits comprise romidepsin and bortezomib. In certain embodiments, given the synergistic interactions between the two pharmaceutical agents, the amount of one or both agents is lower than the amount that is typically administered when the agent is administered alone. In certain embodiments, the amount of both agents is lower. In certain embodiments, the amount administered is sufficient to achieve nanomolar levels in the bloodstream of the subject. In certain embodiments, the amount administered is sufficient to achieve nanomolar concentrations at the site of the cancer or other neoplasm in the subject. The dosing of each of romidepsin and bortezomib is described in more detail above.

As discussed above, the present invention provides novel combinations of romidepsin and a proteasome inhibitor having cytotoxic activity, and thus the inventive compounds are useful for the treatment of a variety of medical conditions including cancer and other neoplasms. In certain embodiments, the agents act synergistically to kill cancer cells. In other embodiments, the agents act additively to kill cancer cells.

The inventive pharmaceutical compositions, preparations, or kits may include other therapeutic agents. The other pharmaceutical agent may be any other therapeutic agent that would be useful to administer to the subject. The other therapeutic agent preferably does not interact adversely with romidepsin or the proteasome inhibitor being administered In certain embodiments, the invention provides for the administration of romidepsin and a proteasome inhibitor in combination with one or more other therapeutic agents, e.g., another cytotoxic agent, steroidal agent, analgesic, etc. In certain embodiments, the other therapeutic agent is another chemotherapeutic agent. In certain embodiments, the other therapeutic agent is a steroidal agent (e.g., prednisone, dexamethasone, prednisolone). The other therapeutic agent may include an agent for alleviating or reducing the side effects of romidepsin and/or the proteasome inhibitor. In certain embodiments, the other therapeutic agent is an anti-inflammatory agent such as aspirin, ibuprofen, acetaminophen, etc., pain reliever, anti-nausea medication, or anti-pyretic. In certain embodiments, the other therapeutic agent is an agent to treat gastrointestinal disturbances such as nausea, vomiting, stomach upset, and diarrhea. These additional agents may include anti-emetics, anti-diarrheals, fluid replacement, electrolyte replacement, etc. In certain particular embodiments, the other therapeutic agent is an electrolyte replacement or supplementation such as potassium, magnesium, and calcium, in particular, potassium and magnesium. In certain embodiments, the other therapeutic agent is an anti-arrhythmic agent. In certain embodiments, the other therapeutic agent is a platelet booster, for example, an agent that increases the production and/or release of platelets. In certain embodiments, the other therapeutic agent is an agent to boost the production of blood cells such as erythropoietin. In certain embodiments, the other therapeutic agent is an agent to prevent hyperglycemia. In certain embodiments, the other therapeutic agent is an immune system stimulator. In certain embodiments, the invention does not include the administration of another HDAC inhibitor besides romidepsin.

It will also be appreciated that certain of the agents utilized in accordance with the present invention can exist in free form for treatment, or where appropriate, as a pharmaceutically acceptable form thereof. According to the present invention, a pharmaceutically acceptable form includes, but is not limited to, pharmaceutically acceptable salts, esters, salts of such esters, protected forms, stereoisomers, isomers, reduced forms, oxidized forms, tautomers, or any other adduct or derivative which upon administration to a patient in need is capable of providing, directly or indirectly, an agent as otherwise described herein, or a metabolite or residue thereof, e.g., a prodrug.

As used herein, the term “pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19, 1977; incorporated herein by reference. The salts can be prepared in situ during the final isolation and purification of the compounds of the invention, or separately by reacting the free base functionality with a suitable organic or inorganic acid. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

Additionally, as used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates, and ethylsuccinates. In certain embodiments, the esters are cleaved by enzymes such as esterases.

Furthermore, the term “pharmaceutically acceptable prodrugs” as used herein refers to those prodrugs of the compounds utilized in accordance with the present invention which are, within the scope of sound medical judgment, suitable for use in contact with the tissues of humans and other animals with undue toxicity, irritation, allergic response, and the like, commensurate with a reasonable benefit/risk ratio, and effective for their intended use. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier or excipient, which, as used herein, includes any and all solvents, diluents, or other liquid vehicles, dispersion or suspension aids, surface active agents, isotonic agents, thickening or emulsifying agents, preservatives, solid binders, lubricants, permeation enhancers, solubilizing agents, and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the anti-cancer compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, hydroxymethylcellulose, hydroxypropylmethylcellulose, methylcellulose, ethylcellulose, and cellulose acetate; powdered tragacanth; malt; gelatin; talc; Cremophor (polyethoxylated caster oil); Solutol (poly-oxyethylene esters of 12-hydroxystearic acid); excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives, and antioxidants can also be present in the composition, according to the judgment of the formulator.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Combination of Romidepsin and Bortezomib in Multiple Myeloma Cells

Methods. Human myeloma U266 and RPM18226 cells were exposed for 48 hours to 2-3 nM romidepsin (FK228) in the absence or presence of 3-4 nM VELCADE® (bortezomib), after which the percentage of apoptotic cells was determined by Annexin V⁺ staining and flow cytometry. Primary CD138⁺ myeloma cells were isolated from a bone marrow sample of a patient with multiple myeloma. CD138⁺ and CD138⁻ cells were then treated for 24 hours with 3 nM romidepsin±3 nM bortezomib, after which apoptosis was assessed by Annexin V-FITC staining and flow cytometry. Dexamethasone-sensitive (MM.1S) and -resistant (MM.1R) human myeloma cells were exposed for 24 hours to 1 nM romidepsin±2 nM bortezomib, after which the percentage of apoptotic cells was determined by Annexin V+staining and flow cytometry. Bortezomib-resistant cells (U266/PS-R) were generated by culturing U266 cells in gradually increasing concentrations of bortezomib until a concentration of 12 nM was reached. U266/PS-R cells were then treated for 48 hours with 2 nM romidepsin in the absence or presence of 5-15 nM bortezomib, after which apoptosis was assessed by Annexin V-FITC staining and flow cytometry. Human multiple myeloma (U266) cells were exposed for 48 hours to 2 nM romidepsin±3 nM bortezomib, after which immunoblot analysis was performed to monitor JNK activation and expression of NF-κB-dependent anti-apoptotic proteins.

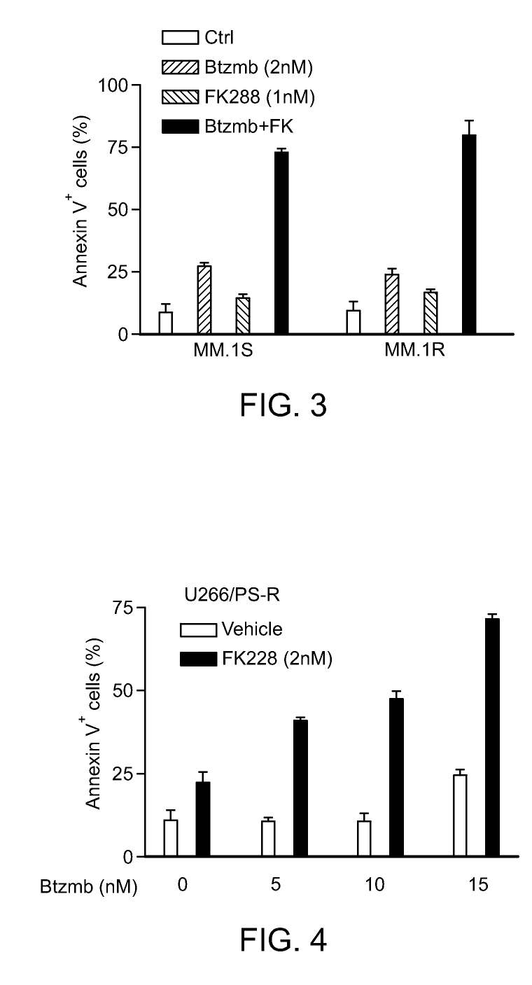

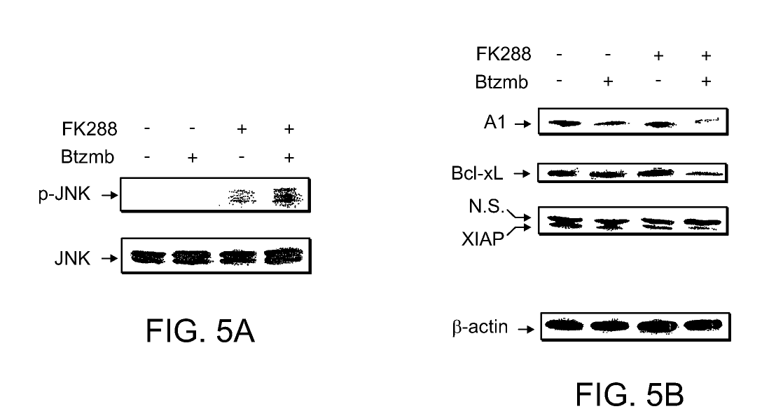

Results. Bortezomib and romidepsin administered at extremely low (nanomolar) concentrations interact in a synergistic manner to induce apoptosis in human multiple myeloma cells lines (FIG. 1). Furthermore, the combination of bortezomib and romidepsin administered at extremely low concentrations induce apoptosis in primary human multiple myeloma cells while sparing normal bone marrow cells, suggesting a possible basis for therapeutic selectivity (FIG. 2). Romidepsin is thought to potentiate bortezomib lethality in steroid-resistant multiple myeloma cells when the two agents are administered at extremely low (nanomolar) concentrations (FIG. 3). Romidepsin also potentiates bortezomib lethality in multiple myeloma cells resistant to bortezomib alone (FIG. 4). Synergistic interactions between romidepsin and bortezomib in human multiple myeloma cells is associated with activation of the stress-related JNK pathway and down-regulation of several NF-κB-dependent anti-apoptotic proteins (e.g., A1, Bcl-xL, and XIAP) (FIG. 5).

Conclusion. Romidepsin and bortezomib, administered concurrently at extremely low concentrations (e.g., low nM) interact in a synergistic manner to induce apoptosis in cultured and primary multiple myeloma cells, including those resistant to steroids or bortezomib. These effects are associated with activation of the stress-related JNK pathway and down-regulation of NF-κB-dependent anti-apoptotic proteins. These findings demonstrate that combination regimens involving romidepsin and bortezomib may be an effective strategy in treating patients with multiple myeloma.

### Example 2

### Combination of Romidepsin and Bortezomib in Chromic Lymphocytic Leukemia (CLL) Cells

Methods. Primary CLL cells were isolated from five CLL patients and exposed for 24 hours to minimally toxic concentrations of bortezomib (3 nM)±romidepsin (3-5 nM), after which cell death was determined by 7AAD staining/flow cytometry and Wright-Giemsa stained cytospin slides under light microscopy. Immunoblot analysis (30 μg of protein per condition) was performed to detect cleavage of caspase-12 and PARP, levels of IκB c, phospho-IκBα, p65, p100/p52, as well as expression of NF-κB downstream anti-apoptotic proteins. Parallel studies were performed in two established CLL cell lines (JVM-3 and MEC-2, DSMZ). Cells were co-treated for 48 hours with sub-toxic concentrations of bortezomib (JVM-3, 3 nM; MEC-2, 5 nM) and romidepsin (JVM-3, 3 nM; MEC-2, 5 nM), after which the percentage of apoptotic cells and cells with loss of mitochondrial membrane potential (Δψm) was determined by Annexin V/PI or DiOC₆ staining, respectively, followed by flow cytometry. Synergism between these agents was evaluated by Median Dose Effect analysis using a commercially available software program (Calcusyn; Biosoft) following administration of bortezomib and romidepsin at a fixed concentration ratio (1:1). Transient transfections were performed using an Amaxa Nucleofector Device (program U-15), and Human B Cell Nucleofector Kit was employed to assess NF-κB activity and functional effects of inactive RelA/p65 mutants (acetylation site mutants).

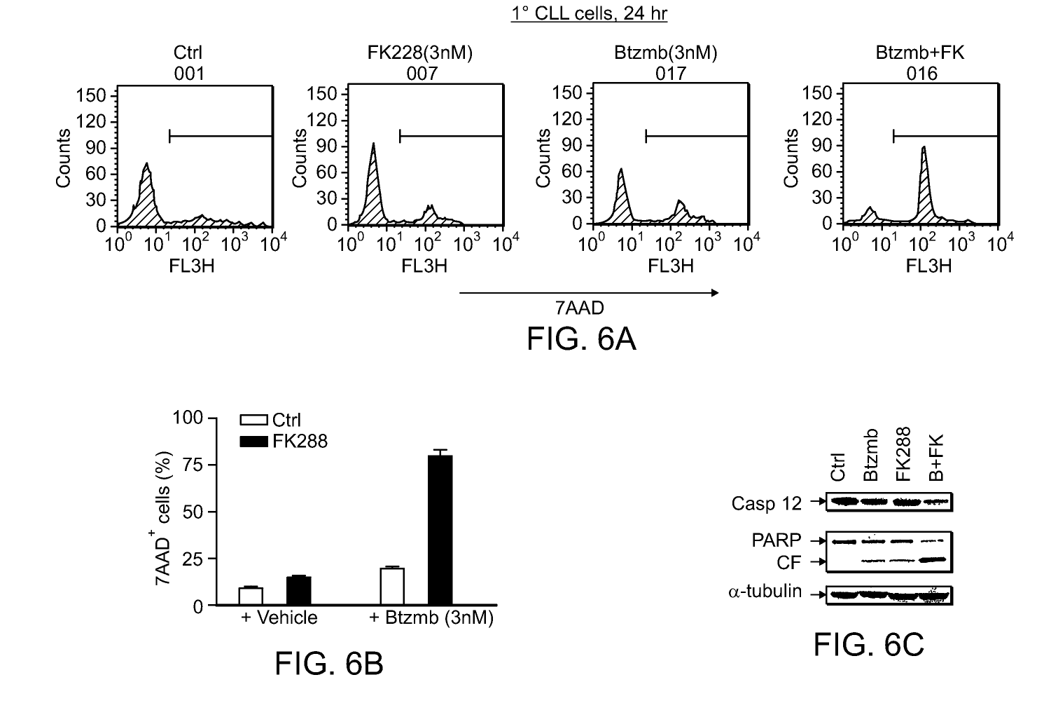

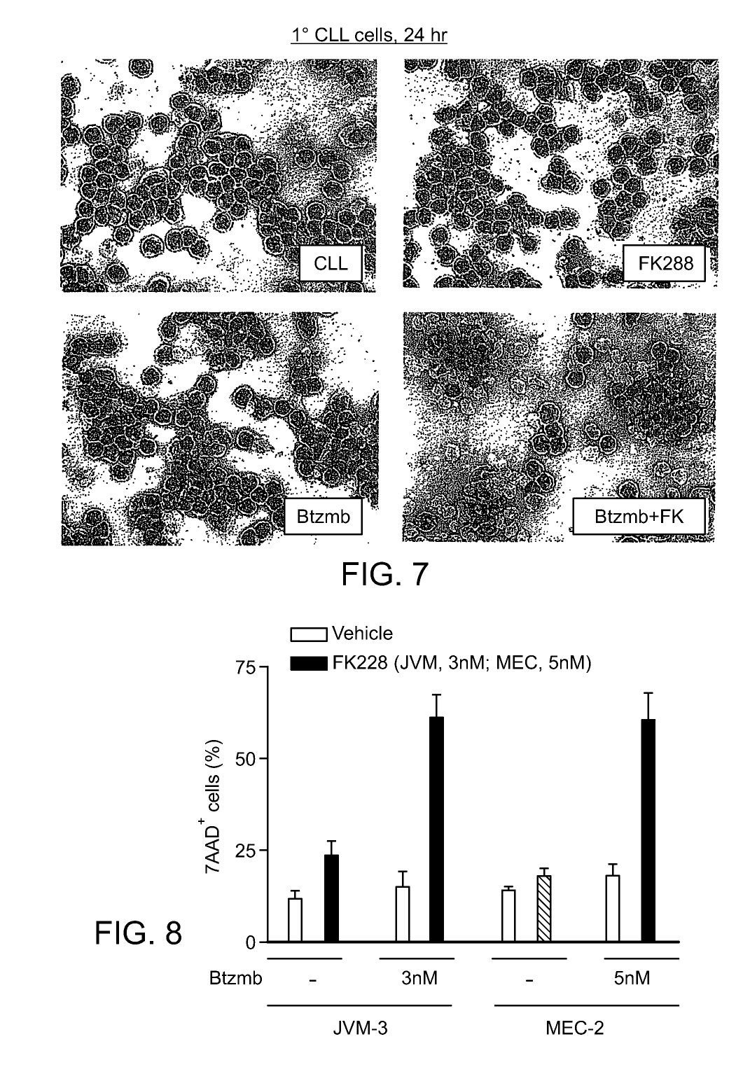

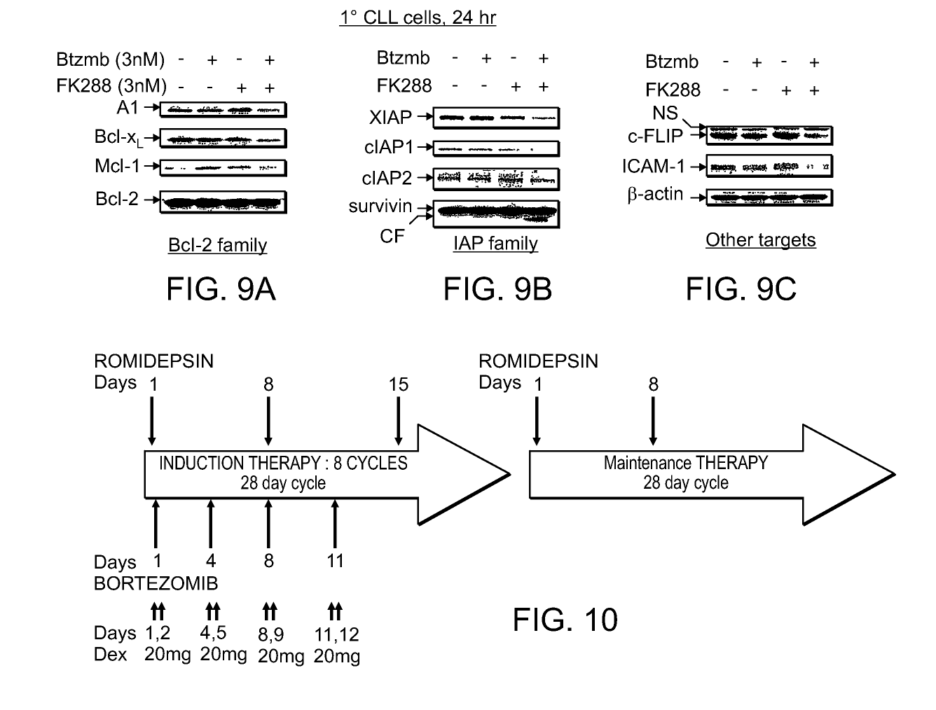

Results. Combined treatment with bortezomib and romidepsin administered at low concentrations (3 nM) potently induces apoptosis and caspase-12 cleavage in primary CLL cells (FIG. 6). Romidepsin dramatically increased bortezomib lethality in four out of five primary CLL samples and resulted in additive lethality in one sample (FIG. 7). Co-administration of romidepsin and bortezomib at subtoxic concentrations (e.g., 2-5 nM) dramatically induced apoptosis in CLL cell lines (JVM-3 and MEC-2). Highly synergistic interactions were documented by Median Dose Effect analysis (FIG. 8). Romidepsin alone activated NF-κB activity in primary CLL cells, an effect that was abrogated by co-administration of bortezomib. Bortezomib also blocked romidepsin-mediated processing of NF-κB2 precursor p100 into its active p52 form. Co administration of bortezomib and romidepsin down-regulated expression of multiple NF-κB downstream survival proteins including A1, Bcl-xL, XAIP, cIAP1, cIAP2, c-FLIP, and ICAM-1. Bortezomib treatment alone induced accumulation of Mcl-1, which was diminished in cells co-exposed to romidepsin. The combination also resulted in cleave of the anti-apoptotic protein survivin (FIG. 9).

Conclusion. Co-administration of marginally toxic concentrations of romidepsin and bortezomib results in the highly synergistic induction of apoptosis in primary human CLL cells and CLL cell lines. These events are associated with disruption of the NF-κB pathway and down-regulation of multiple anti-apoptotic members of the Bcl-2 family. Collectively, these findings raise the possibility that combination regimens involving romidepsin and bortezomib may be an effective strategy in treating patients with refractory CLL.

### Example 3

### Treatment of Multiple Myeloma Using Combination of Romidepsin and Bortezomib

The combination of romidepsin and bortezomib was used to treat patients with multiple myeloma (MM) in a human clinical trial. The clinical trial was an open label, single-center, single-arm, phase I/II dose escalation trial of bortezomib, dexamethasone, and romidepsin (depsipeptide, FK228) in patients with relapsed or refractory multiple myeloma, followed by maintenance romidepsin therapy until disease progression. The trial design is illustrated in FIG. 10.

The objectives of the clinical trial include determining the maximum tolerated dose (MTD) of romidepsin administered with bortezomib in patients with relapsed multiple myeloma and the efficacy of this combination at the MTD in terms of overall response, time to progression, and overall survival.

Specifically, during phase I (accelerated dose escalation phase), an accelerated titration design was used to ascertain the MTD of romidepsin. For example, if no dose limiting toxicities (DLTs) and <2 patients have moderate toxicities in cycle 1 (C1), the next patient is entered at one level higher. According to National Cancer Institute Common Toxicity Criteria (NCI-CTC) (version 3), DLT is defined as platelets<25×10⁹/L; Grade 4 neutropenia despite G-CSF support; Grade 3 or 4 nausea, emesis, or diarrhea despite treatment; any other Grade 3 or 4 non-haematological toxicity; or >4 week suspension of treatment due to toxicity. The accelerated phase ends when one patient has a DLT during C1 or two patients have “moderate” toxicity during first treatment cycles. Patients were entered in cohorts of three according to the standard dose escalation design. A maximum of six patients were treated at any dose level. MTD is defined as the highest dose level at which the incidence of DLT is less than 33%. A typical dose escalation schedule is shown in Table 1 below.

TABLE 1
Dose Escalation Schedule

| | Bortezomib | Dexamethasone | Romidepsin |
|---|---|---|---|
| Dose level | (mg/ml²) | (mg) | (mg/ml²) |
| Level 1 | 1.3 | 20 | 8 |
| Level 2 | 1.3 | 20 | 10 |
| Level 3 | 1.3 | 20 | 12 |
| Level 4 | 1.3 | 20 | 14 |

Following the determination of MTD, an additional 15 patients are accrued during phase II at the MTD to obtain further data concerning the efficacy of the bortezomib-romidepsin combination.

Patients eligible enrolment must be 18 years or older who have relapsed multiple myeloma and have up to four prior lines of therapy. Eligible patients should also have measurable disease and are required to have platelet count of 50×10⁹/l or more, blood hemoglobin (Hb) concentration of at least 75 g/L; absolute neutrophil count at least 0.75×10⁰/L; and adequate liver and renal function.

Patients who are not eligible for the trail include those who have neuropathy of grade 3 or worse, or neuropathy of grade 2 with pain of grade 1 or worse according to the criteria set forth by the National Cancer Institute Common Toxicity Criteria of Adverse Events (NCI-CTCAE) (version 3.0); who have history of cardiac arrythythmias or active coronary artery disease; and who use concomitant drugs including drugs causing prolongation of QTc interval and/or inhibitors of CYP3A4.

The characteristics of the enrolled patients are summarised in Table 2 below.

TABLE 2
Patient Characteristics

| | N = 10 |
|---|---|
| Patient Characteristics | |
| Male | 3 |
| Median age (year, range) | 62 (40-78) |
| Median no. of previous line of treatment | 2 (1-5) |
| Past treatments: | |
| VAD | 6 |
| HDT + AuSCT | 6 |
| Oral melphalan | 3 |
| Cyclophosphamide | 4 |
| Thalidomide +/− Dexamethasone | 4 |
| Revlimid + Dexamethasone | 1 |
| DTPACE | 1 |
| Interferon-α | 1 |

All patients received bortezomib at a dose of 1.3 mg/m² on days 1, 4, 8, and 11, and dexamethasone at a dose of 20 mg on days 1, 2, 4, 5, 8, 9, 11, and 12 of a 28-day cycle. The dose escalation of romidepsin commenced at a dose of 8 mg/m² intravenously on days 1, 8, and 15 of the 28-day cycle and involved an initial accelerated dose escalation phase, with intra-patient dose escalation of romidepsin.

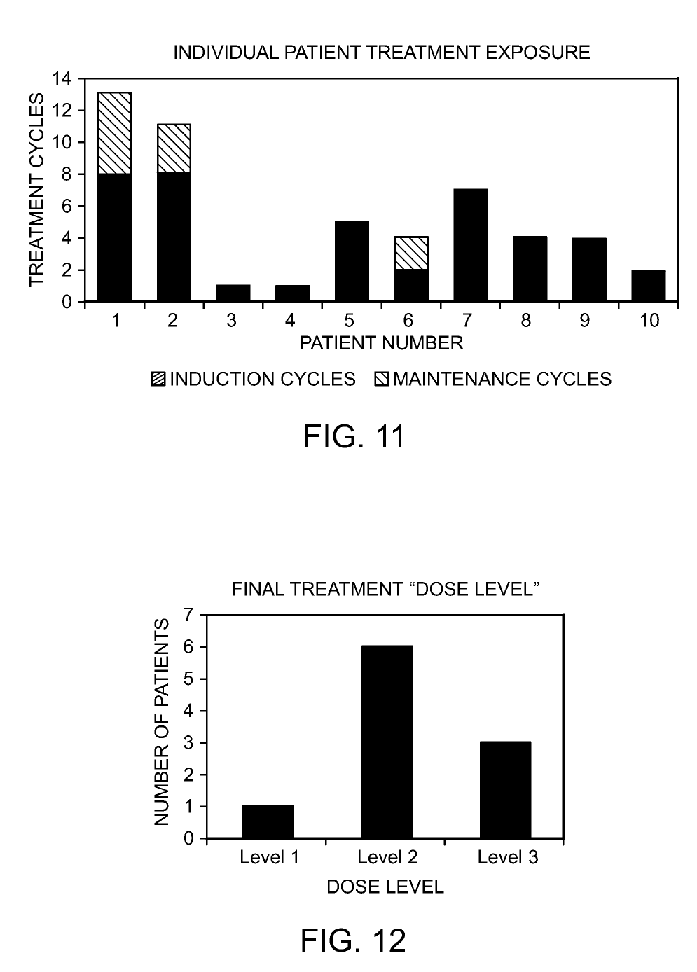

This 28-day induction cycle can be repeated, for example, up to 8 cycles. As of August 2007, the median number of cycles delivered to the patients was 3. The number of cycles that each individual received is summarised in FIG. 11. The distribution of the final treatment dose level is shown in FIG. 12.

To date, no dose-limiting toxicities were demonstrated at romidepsin doses of 8 mg/m² (n=1) or 10 mg/m² (n=3). At the romidepsin dose of 12 mg/m², three episodes of Grade 4 thrombocytopenia and one episode of febrile neutropenia occurred. Of note, two of the patients with Grade 4 thrombocytopaenia had platelets below 100×10⁹/L prior to commencing the combination (at inclusion, patients must have had a platelet count>50×10⁹/L).

Other drug-related toxicities observed include: Grade 3 fatigue (n=1); neutropaenia (n=1); sepsis (n=1); Grade 2 fatigue (n=1); peripheral neuropathy (n=2); nausea (n=1); and diarrhea (n=1). Two patients required dose reduction of bortezomib due to peripheral neuropathy (these patients were co-administered romidepsin at 12 mg/m²). Table 3 summarises drug-related adverse events.

TABLE 3
Drug-related adverse events

| Adverse events (n = 10 Pts) | All grade | Grade 3/4 | DLT |
|---|---|---|---|
| Nausea | 8 | 0 | |
| Fatigue | 7 | 2 | |
| Constipation/diarrhoea | 7 | 1 | 1 |
| Neuropathy (sensory or motor) | 6 | 1 | 1 |
| Infection | 4 | 2 | |
| Oedema | 4 | 0 | |
| Thrombocytopenia | 6 | 4 | 1 |
| Anaemia | 4 | 2 | |
| Transaminitis | 2 | 1 | |
| Anorexia | 3 | 0 | |
| Infections | 4 | 2 | 1 |
| Febrile neutropenia | 2 | 0 | 1 |
| Other | 2 | 1 | 1 |

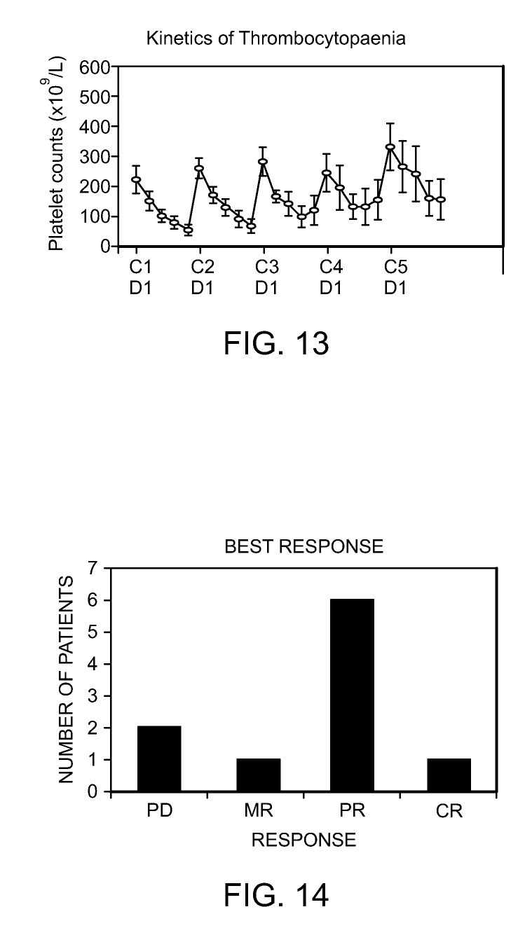

The bortezomib and romidepsin combination was well tolerated. The maximum tolerated dose corresponds to the treatment dose level 2, i.e., bortezomib at a dose of 1.3 mg/m², dexmethasone at a dose of 20 mg, and romidepsin at a dose of 10 mg/m². As indicated in Table 3, thrombocytopenia is the most common grade≧3 toxicity. Exemplary kinetics of thrombocytopenia is illustrated in FIG. 13.

Response was assessed on day 1 of cycle 3, 5, and 7 and day 1 for each maintenance cycle. Response rates were assessed according to M-protein response criteria, with complete responses documented by Blood and Marrow Transplantation (EMBT) criteria. Table 4 summarises the response results of the trial. There were 1 immunofixation negative Complete Response (CR), 6 Partial Responses (PR) and 1 Minor Response (MR) among the patients (also see FIG. 14). Most patients remain on the combination therapy. The trial continues accruing patients at the doses of romidepsin (10 mg/m²) with bortezomib (1.3 mg/m²).

TABLE 4
Response results of the trial

| | | | % M-protein | | | Time to |
|---|---|---|---|---|---|---|
| | Time on | | reduction. | Time to a | | progres- |
| | therapy | | (November | response | | sion |
| Patient | (months) | Response | 2007) | (months) | | (months) |
| 1 | 12+ | CR (IF neg) | 100 | 1 | | NA |
| 2 | 10 | PR | 98 | 2 | | 10 |
| 3 | 1 | PD | NA | NA | | <1 |
| 4 | 1 | PD | NA | NA | | <1 |
| 5 | 5 | MR (*) | 42 | 1.7 | (*) | 5 |
| 6 | 6+ | PR | 75 (Φ) | 1 | (Φ) | NA |

| | | | | | |
|---|---|---|---|---|---|
| 7 | 6+ | PR | 57 | 1.7 | NA |
| 8 | 3+ | PR | 89 | 2 | NA |
| 9 | 3+ | PR | 75 | 1 | NA |
| 10 | 2+ | PR | 88 | 0.7 | NA |

+Patient still on therapy.
(*) MR: minor response.
(Φ) Patient with non-secretory multiple myeloma; response based on reduction of bone marrow plasma cell infiltrate.

The results of the clinical trial demonstrate that the combination of bortezomib and romidepsin shows a promising response rate and durable responses in relapsed/refractory multiple myeloma.

### EQUIVALENTS AND SCOPE

The foregoing has been a description of certain non-limiting preferred embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a”, “an”, and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For example, in certain embodiments of the invention the biologically active agent is not an anti-proliferative agent. For purposes of brevity, all of the embodiments in which one or more elements, features, purposes, or aspects is excluded are not set forth explicitly herein.

1. A method of treating cancer or other neoplasm in a subject, the method comprising steps of:
administering a therapeutically effective amount of romidepsin and a proteasome inhibitor to a subject with cancer or other neoplasm.

2. The method of claim 1, wherein romidepsin is of the formula:

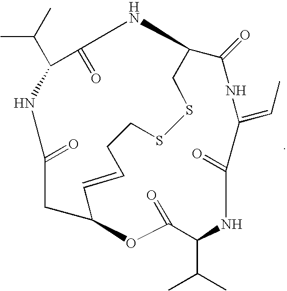

3. The method of claim 1, wherein the proteasome inhibitor is selected from the group consisting of bortezomib (VELCADE®), peptide boronates, salinosporamide A (NPI-0052), lactacystin, epoxomicin (Ac(Me)-Ile-Ile-Thr-Leu-EX), MG-132 (Z-Leu-Leu-Leu-al), PR-171, PS-519, eponemycin, aclacinomycin A, CEP-1612, CVT-63417, PS-341 (pyrazylcarbonyl-Phe-Leu-boronate), PSI (Z-Ile-Glu(OtBu)-Ala-Leu-al), MG-262 (Z-Leu-Leu-Leu-bor), PS-273 (MNLB), omuralide (clasto-lactacystin-β-lactone), NLVS (Nip-Leu-Leu-Leu-vinyl sulfone), YLVS (Tyr-Leu-Leu-Leu-vs), dihydroeponemycin, DFLB (dansyl-Phe-Leu-boronate), ALLN (Ac-Leu-Leu-Nle-al), 3,4-dichloroisocoumarin, 4-(2-aminoethyl)-benzenesulfonyl fluoride, TMC-95A, gliotoxin, EGCG ((−)-epigallocatechin-3-gallate), and YU101 (Ac-hFLFL-ex).

4. The method of claim 1, wherein the proteasome inhibitor is bortezomib (VELCADE®).

5-7. (canceled)

8. The method of claim 1, wherein the cancer is a malignancy of hematological cells.

9. The method of claim 1, wherein the cancer is a leukemia.

10-14. (canceled)

15. The method of claim 1, wherein the cancer is a lymphoproliferative malignancy.

16. The method of claim 1, wherein the cancer is multiple myeloma.

17. The method of claim 1, wherein the cancer is plasma cell-derived cancer.

18-20. (canceled)

21. The method of claim 1, wherein the cancer is a solid tumor.

22. (canceled)

23. The method of claim 1, wherein the cancer is a relapsed cancer.

24. The method of claim 1, wherein the cancer is a refractory cancer.

25. The method of claim 1, wherein the cancer is a bortezomib (VELCADE®)-resistant cancer.

26. The method of claim 1, wherein the cancer is a steroid-resistant cancer.

27. The method of claim 1, wherein the cancer is dexamethasone-resistant multiple myeloma.

28. The method of claim 1, wherein the therapeutically effective amount of romidepsin ranges from approximately 0.5 mg/m² to approximately 28 mg/m².

29. (canceled)

30. (canceled)

31. The method of claim 1, wherein the therapeutically effective amount of romidepsin ranges from approximately 8 mg/m² to approximately 14 mg/m².

32. (canceled)

33. (canceled)

34. The method of claim 1, wherein the therapeutically effective amount of romidepsin is approximately 10 mg/m².

35. (canceled)

36. (canceled)

37. The method of claim 4, wherein the therapeutically effective amount of bortezomib (VELCADE®) ranges from approximately 0.1 mg/m² to approximately 5 mg/m².

38-40. (canceled)

41. The method of claim 4, wherein the therapeutically effective amount of bortezomib (VELCADE®) ranges from approximately 0.75 mg/m² to approximately 1.5 mg/m².

42. (canceled)

43. The method of claim 4, wherein the therapeutically effective amount of bortezomib (VELCADE®) is approximately 1.3 mg/m².

44. The method of claim 4, wherein the therapeutically effective amount of romidepsin ranges from 4 mg/m² to 15 mg/m²; and wherein the therapeutically effective amount of bortezomib (VELCADE®) ranges from 0.5 mg/m² to 3 mg/m².

45. (canceled)

46. (canceled)

47. The method of claim 1 further comprising administering another anti-neoplastic agent.

48. The method of claim 1 further comprising administering a cytotoxic agent.

49. The method of claim 1 further comprising administering a steroidal agent.

50. The method of claim 49, wherein the steroidal agent is selected from the group consisting of alclometasone diproprionate, amcinonide, beclomethasone diproprionate, betamethasone, betamethasone benzoate, betamethasone diproprionate, betamethasone sodium phosphate, betamethasone sodium phosphate and acetate, betamethasone valerate, clobetasol proprionate, clocortolone pivalate, cortisol (hydrocortisone), cortisol (hydrocortisone) acetate, cortisol (hydrocortisone) butyrate, cortisol (hydrocortisone) cypionate, cortisol (hydrocortisone) sodium phosphate, cortisol (hydrocortisone) sodium succinate, cortisol (hydrocortisone) valerate, cortisone acetate, desonide, desoximetasone, dexamethasone, dexamethasone acetate, dexamethasone sodium phosphate, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium succinate, mometasone furoate, paramethasone acetate, prednisolone, prednisolone acetate, prednisolone sodium phosphate, prednisolone tebutate, prednisone, triamcinolone, triamcinolone acetonide, triamcinolone diacetate, and triamcinolone hexacetonide, and combinations thereof.

51. (canceled)

52. The method of claim 50, wherein the steroidal agent is dexamethasone.

53-54. (canceled)

55. The method of claim 52, wherein dexamethasone is administered at a dose ranging from 10 mg to 50 mg.

56. (canceled)

57. The method of claim 52, wherein dexamethasone is administered at a dose of approximately 20 mg.

58. The method of claim 1, wherein romidepsin and the proteasome inhibitor are administered intravenously.

59. (canceled)

60. The method of claim 59, wherein romidepsin is administered weekly and the proteasome inhibitor is administered twice a week.

61. (canceled)

62. The method of claim 49, wherein the steroidal agent is administered together with the romidepsin or the proteasome inhibitor.

63. The method of claim 49, wherein the steroidal agent is administered prior to or following the administration of romidepsin or the proteasome inhibitor.

64. (canceled)

65. A method of treating multiple myeloma in a subject, the method comprising steps of:
administering a therapeutically effective amount of romidepsin and bortezomib to a subject with multiple myeloma.

66. (canceled)

67. The method of claim 66, wherein the therapeutically effective amount of romidepsin ranges from 8 mg/m² to 10 mg/m².

68. The method of claim 65, wherein the therapeutically effective amount of bortezomib (VELCADE®) ranges from 0.5 mg/m² to 3 mg/m².

69. The method of claim 68, wherein the therapeutically effective amount of bortezomib (VELCADE®) is approximately 1.3 mg/m².

70. The method of claim 65, wherein the therapeutically effective amount of romidepsin ranges from 8 mg/m² to 10 mg/m²; and wherein the therapeutically effective amount of bortezomib (VELCADE®) is approximately 1.3 mg/m².

71. The method of claim 70, wherein romidepsin is administered weekly and bortezomib (VELCADE®) is administered twice a week.

72. The method of claim 65, wherein the method further comprises administering a steroidal agent.

73. The method of claim 72, wherein the steroidal agent is selected from the group consisting of alclometasone diproprionate, amcinonide, beclomethasone diproprionate, betamethasone, betamethasone benzoate, betamethasone diproprionate, betamethasone sodium phosphate, betamethasone sodium phosphate and acetate, betamethasone valerate, clobetasol proprionate, clocortolone pivalate, cortisol (hydrocortisone), cortisol (hydrocortisone) acetate, cortisol (hydrocortisone) butyrate, cortisol (hydrocortisone) cypionate, cortisol (hydrocortisone) sodium phosphate, cortisol (hydrocortisone) sodium succinate, cortisol (hydrocortisone) valerate, cortisone acetate, desonide, desoximetasone, dexamethasone, dexamethasone acetate, dexamethasone sodium phosphate, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium succinate, mometasone furoate, paramethasone acetate, prednisolone, prednisolone acetate, prednisolone sodium phosphate, prednisolone tebutate, prednisone, triamcinolone, triamcinolone acetonide, triamcinolone diacetate, and triamcinolone hexacetonide or a synthetic analog thereof, or a combination thereof.

74. (canceled)

75. The method of claim 73, wherein the steroidal agent is dexamethasone.

76-77. (canceled)

78. The method of claim 75, wherein dexamethasone is administered at a dose of approximately 20 mg.

79. (canceled)

80. The method of claim 79, wherein romidepsin is administered weekly and the bortezomib (VELCADE®) is administered twice a week.

81-82. (canceled)

83. The method of claim 72, wherein the steroidal agent is administered prior to or following the administration of romidepsin or the bortezomib (VELCADE®).

84. (canceled)

85. A method of treating cells, the method comprising steps of:
administering a combination of romidepsin and bortezomib (VELCADE®) to a cell.

86. The method of claim 85, wherein the step of administering comprises administering a combination of romidepsin and bortezomib (VELCADE®) to a cell at a concentration sufficient to kill the cell.

87-102. (canceled)

103. A method of inducing apoptosis in a cell, the method comprising:
administering an amount of romidepsin and bortezomib effective to induce apoptosis in a cell.

104-107. (canceled)

108. A pharmaceutical composition for treating cancer comprising a therapeutically effect amount of romidepsin, and a therapeutically effective amount of bortezomib.

109. (canceled)

110. (canceled)

111. The pharmaceutical composition of claim 108, wherein the cancer is multiple myeloma.

112-114. (canceled)
